(19) United States
(12) Patent Application Publication
(10) Pub. No.: US 2022/0313977 A1
(43) Pub. Date: Oct. 6, 2022

(54) PRIMING APPARATUS FOR A DRIP CHAMBER OF A FLUID INFUSION SYSTEM

(71) Applicant: Mobile I.V. Systems, LLC, Chugiak, AK (US)

(72) Inventors: Gerold Gugel, Chugiak, AK (US); Ryan Gessler, Camas, WA (US); Dale Constuble, Helendale, CA (US)

(73) Assignee: Mobile I.V. Systems, LLC, Chugiak, AK (US)

(21) Appl. No.: 17/630,149

(22) PCT Filed: Jul. 27, 2020
(86) PCT No.: PCT/US2020/043776

§ 371 Date: Jun. 6, 2022

Related U.S. Application Data
(60) Provisional application No. 62/879,262, filed on Jul. 26, 2019.

(51) Int. Cl. A61M 39/20; A61M 5/14; A61M 5/38

(52) U.S. Cl. CPC A61M 39/20; A61M 5/1411; A61M 5/385; A61M 2005/1402; A61M 2039/205; A61M 2039/2486

(57) ABSTRACT

Apparatuses for priming a drip chamber of an infusion tube seta are described. A priming apparatus may be incorporated into a cavity of a body, such as a drip chamber cap, that is attached to or integrally formed with the inlet side of drip chamber. The priming apparatus includes a closure mechanism at least a portion of which is accommodated in a cavity in the body. The cavity is part of an outflow or vent passage through the body (e.g., the drip chamber cap) and the closure mechanism is operable, e.g., responsive to user force, to selectively open the outflow passage, thereby unsealing the vent cavity to ambient air and allowing air to be purged from the fluid system during priming of the drip chamber. When the closure mechanism is an closed position, the outflow passage and vent cavity as sealed from ambient air, preventing any air from passing into the fluid system through the cap.

### CROSS-REFERENCE TO RELATED APPLICATION(S)

This application claims priority to U.S. Provisional Application No. 62/879,262 filed Jul. 26, 2020, which is incorporated herein by reference, in its entirety, for any purpose.

### TECHNICAL FIELD

This application describes an apparatus for priming the drip chamber of an intravenous fluid infusion tube set.

### BACKGROUND

Intravenous (IV) therapy refers to the delivery of a liquid substance (e.g., a glucose solution, saline solutions, medication in liquid form, an aqueous physiologically-acceptable fluid, and blood or plasma) directly into a vein. IV therapy can be administered via an injection (with a syringe at higher pressure) or via infusion, which typically uses the pressure supplied by gravity and is commonly referred to as an IV drip. An IV line is most commonly set up as a peripheral line (PIV), with the fluid delivered into a peripheral vein such as in an arm, hand, leg or foot of the patient. Contraindications for a peripheral line may require the placement of a central IV line, which delivers fluid into a large central vein in the torso such as the inferior or superior vena cava. Alternatively, fluids may be delivered via interosseous infusion, which involves infusion directly into the marrow of a long bone of the upper arm or leg.

Setting up a peripheral IV (PIV) line typically involves the insertion of a peripheral venous catheter (PVC), cannula, or large gauge needle in a peripheral vein and connecting the infusion tubing/administration tube set (also referred to simply as IV tube set) to the PVC, cannula or needle. For the administration of fluids to a human patient, IV tube sets are typically categorized as either macro-drip sets (e.g., that deliver anywhere between 10-20 drops/mL), typically used for adult patients, or micro drip sets (e.g., that deliver around 60 drops/mL), typically used in pediatric or neonatal care. IV fluids may also be administered to non-human subjects or patients, for example in the veterinary filed. The administration of IV fluids may be desirable in a variety of circumstances, for example in the course of medical treatment or prophylactically, such as to speedup recovery and prevent dehydration after exertion. IV tube sets typically include IV tubing, a spike for connecting the IV tubing to the IV bag, and a drip chamber which enables the medical professional to monitor the rate of administering fluids. The IV tubing is typically flexible clear tubing that may be equipped with a check valve, one or more access ports (e.g., for delivering secondary medication), a roller clamp and optionally secondary tubing which may be connected to the primary tubing via a Y-port or Y-site.

While IV therapy has been widely available since the mid-1900s, commercially available IV infusion sets have remained mostly unchanged for decades and the risks and inefficiencies associated with them remaining mostly unaddressed. For example, a significant risk associated with IV therapy is air embolism, which can result from air passing through the fluid line into the circulatory system of the patient and causing a blood vessel blockage. Set up and monitoring procedures, when using conventional tube sets, such as the priming of the tube set before connecting to the patient, keeping the IV bag at least 3 feet above the insertion site and the drip chamber vertical at all times, all of which may be necessary to reduce the risk to the patient, may be difficult to follow, require additional equipment and personnel, and cause delay in patient care, particularly in emergency response scenarios. In high risk scenarios such as on a battlefield, during a terrorist attack or in active shooter emergencies, etc., these requirements can put caregivers and patients at needless risk of exposure to the dangers. Moreover, requirements as to orientation and position of a conventional drip chamber for proper/safe use may impose ambulatory restrictions on the patient or subject which may be undesirable or difficult to achieve. Thus, designers and manufacturers of IV tube sets continue to seek improvements thereto and embodiments described herein may address some of the limitations of existing solutions.

### SUMMARY

Generally, the embodiments described herein relate to an IV fluid infusion system that includes an IV bag, and more specifically an apparatus for purging air out of a fluid infusion system (e.g., out of the bag and drip chamber) while priming the drip chamber. IV fluid is often administered from a flexible container or bag containing the intravenous (IV) fluid (e.g., saline-based IV solution, a fluid containing medication, and/or a blood-based product such as plasma). An IV bag typically contains some amount of air or other gas sealed within the bag that holds the IV fluid, and which remains in or is added to during administration of the fluid. The type of gas within the bag may depend upon the type of fluid, however the term air will be used throughout to refer to air or any other type of gas that may be in the fluid infusion system (e.g., in the IV bag). This air allows volume to be read via the fluid meniscus when the bag is hung vertically. Apparatuses according to the present examples enable purging substantially all of the air from the IV bag during the priming process, which reduces the amount of air in the fluid system upstream of the drip chamber outlet, as well as prevent any air from being added to the fluid system (e.g., the IV bag), thereby reducing the risk of air passing into a blood vessel of the subject (e.g. human or non-human patient). While not so limited, the examples herein are well suited for use with a pressurized fluid delivery system whereby external pressure (e.g., applied by a pressure cuff or manually) is applied to the IV bag to facilitate the flow of fluid out of the bag and into the drip chamber.

In some embodiments, an apparatus for priming a drip chamber of an infusion tube set includes a body having an inlet, an outlet, and a fluid passage connecting the inlet to the outlet. The fluid passage may be implemented by a single or a plurality of passages extending from the inlet to the outlet of the body. The body is configured to be coupled to a drip chamber to position the outlet in fluid communication with an interior of the drip chamber. In some embodiments, body configured to be provided across the inlet of a drip chamber to seal the drip chamber inlet. The apparatus may thus function as, and be referred to as a drip chamber cap. When the drip chamber cap is connected to the drip chamber, the fluid passage allows IV fluid to be transmitted from the bag into the drip chamber. The body may further define a secondary (or vent) passage through the cap which is used to draw or vent air out of the fluid system during priming of the system (e.g., the drip chamber). A means for hermetically sealing and thus preventing flow of air into or out of the fluid system through the cap. The means may be operatively associated with the secondary vent passage and may be operated by a user. The means may be implemented by a valve that includes a closure mechanism configured to allow and block the flow of ambient air into a cavity in the body that forms part of the secondary (vent) passage. In some embodiments of the apparatus, the closure mechanism is received at least partially in the cavity, the cavity being in fluid communication with the interior of the drip chamber via a valve inlet and in fluid communication with ambient air via a valve outlet. The valve (e.g., closure mechanism) is configured to be actuated by a user to selectively open and seal the valve outlet, the cavity being hermetically sealed from the ambient air when the valve outlet is sealed. The closure mechanism may be implemented using any suitable means for hermetically sealing the vent cavity from ambient air. The closure mechanism can be actuated by the application of user force on an actuator of the closure mechanism, which engages a sealing member operatively positioned within the cavity to seal the valve outlet and thus hermetically seal the cavity when the closure mechanism is in a closed position.

According to some embodiments, a fluid infusion set includes a drip chamber having a drip chamber inlet for providing a fluid into an interior of the drip chamber and a cap covering the drip chamber inlet, the cap being provided by a cap body defining a cap inlet on a distal side of the cap body, a cap outlet on a proximal side of the cap body and a fluid passage extending through the cap body and connecting the cap inlet to the cap outlet. In some embodiments, the cap is separately formed from the drip chamber and attached thereto by any suitable means (e.g., via a coupling interface, such as a male-female coupling, that may be press fitted and/or bonded). In some embodiments, the cap (e.g., the cap body) and at least a portion of the drip chamber, for example an upper portion (e.g., upper half) of the drip chamber may be integrally formed.

The fluid infusion set according to embodiments herein may further include a valve received within a cavity in the cap body, wherein the cavity communicates with the interior of the drip chamber via a valve inlet opening on the proximal side of the cap body and wherein the cavity communicates with ambient air via a valve outlet, and wherein the valve includes a closure mechanism actuatable by a user between an open position in which air is permitted to pass through the valve outlet and a closed position in which the cavity is hermetically sealed from the ambient air. The closure mechanism is biased toward the closed position. In some embodiments, the closure mechanism comprises a button and a seal configured to seal the valve outlet when the closure mechanism is in the closed position. The seal may be positioned between a base of the button and the valve outlet. In other embodiments, the button compresses the seal in a direction away from the valve outlet when the valve is in the open position. In some embodiments, the outlet is defined by a central bore in a retainer of the closure mechanism, the retainer received and frictionally engaging a sidewall of the cavity to retain the button and seal in the cavity. In some embodiments, the diameter of the central bore varies along a length of the central bore to accommodate at least a portion of the base within the central bore. In some embodiments, a spike extends from a distal side of the cap body. In some embodiments, the spike is integrated with the cap body. In some embodiments, the fluid infusion set further includes an antimicrobial filter at the valve inlet opening. In some embodiments, the cap body defines an annular bore on the proximal side of the cap body, and wherein the filter is provided in the annular bore. In some embodiments, the filter is attached to a filter support received at least partially within the annular bore. In some embodiments, the filter support includes a central tube aligned with the cap outlet and having a different inner diameter than a diameter of the cap outlet.

In some embodiments, the drip chamber is an all position drip chamber, configured to be used for intravenous fluid delivery in any orientation of the drip chamber. In some embodiments, the drip chamber includes a spherical body portion and a neck portion, and an outlet of the drip chamber is positioned within an interior of the spherical portion to remain submerged in fluid irrespective of orientation of the drip chamber when the drip chamber is filled with fluid to a predetermined fill level. In some embodiments, the drip chamber is substantially rigid, for example made from a substantially rigid plastic material.

This summary is neither intended nor should be construed as being representative of the full extent and scope of the present disclosure. The present disclosure is set forth in various levels of detail in this application and no limitation as to the scope of the claimed subject matter is intended by either the inclusion or non-inclusion of elements, components, or the like in this summary

### BRIEF DESCRIPTION OF THE DRAWINGS

The accompanying drawings, which are incorporated in and constitute a part of the specification, illustrate examples of the disclosure and, together with the general description given above and the detailed description given below, serve to explain the principles of these examples.

FIG. 1 shows an example emboli-reducing drip chamber with a spike cap.

FIG. 2 shows a cross-sectional view of the drip chamber of FIG. 1 taken at line 2-2 in FIG. 1, and showing the priming valve in a first, closed configuration.

FIG. 3 shows a cross-sectional view similar to that of FIG. 2 but with the priming valve in a second, open configuration.

FIG. 4 shows a cross-sectional view of the spike cap of FIG. 1.

FIG. 5 shows an exploded view of the spike cap of FIG. 4.

FIGS. 6A-6C show various configurations of tube sets according to the present disclosure, each including a priming valve incorporated at different locations of the tube set.

FIG. 7 shows an elevation view of a drip chamber cap with a priming valve integrated into the cap body.

FIG. 8 shows a cross-sectional view of the cap of FIG. 7 taken at line 8-8 in FIG. 7.

FIG. 9 shows an elevation view of a spike cap in accordance with further examples of the present disclosure.

FIG. 10 shows a cross-sectional view of the spike cap of FIG. 9 taken at line 10-10 in FIG. 9.

FIG. 11 shows an enlarged partial cross-sectional view of the spike cap in FIG. 10.

FIG. 12 shows an isometric view of a filter retainer;

FIG. 13 shows an elevation view of a spike cap in accordance with further examples of the present disclosure;

FIG. 14 shows a cross-sectional view of the spike cap of FIG. 13 taken at line 14-14 in FIG. 13;

FIG. 15 shows an elevation view of a spike cap that includes a button lock in accordance with further examples of the present disclosure;

FIG. 16 shows the spike cap of FIG. 15 with the button lock in the engaged or locked position.

FIG. 17 shows a spike cap of the present disclosure in combination with a drip chamber of a different form factor.

FIG. 18 shows a modular system for an IV administration tube set including multiple different inflow members, a drip chamber cap incorporating a priming apparatus, drip chamber components for assembling drip chambers of different configurations and a plurality of different outflow members.

FIG. 19 shows an exploded view of a priming apparatus incorporated into a drip chamber cap according to further examples of the present disclosure.

FIG. 20 shows a cross-sectional view of the cap of FIG. 19 taken at line 20-20 in FIG. 19.

FIG. 21 shows a drip chamber assembly with a portion of the cap cut away to show the internal components of the priming valve.

FIG. 22 shows a flow diagram of a process for assembling an infusion tube set according to some examples of the present disclosure.

FIG. 23 shows a flow diagram of a process for priming a drip chamber of an infusion tube set according to some examples of the present disclosure.

FIG. 24 shows another example of a priming apparatus according to the present disclosure.

### DETAILED DESCRIPTION

Generally, the embodiments described herein relate to IV infusion systems, and more particularly to IV tube sets and apparatuses associated with the same. In some embodiments, a fluid infusion set (e.g., an IV tube set) may include a drip chamber, which may be, but need not be, an emboli-reducing drip chamber, and a cap covering the inlet of the drip chamber. In some embodiments, the cap body may be separately formed form the drip chamber (e.g., from the upper portion thereof) and suitably attached to the drip chamber to cover the drip chamber inlet. In other embodiments, the cap (e.g., cap body) is integrally formed with at least a portion of the drip chamber (e.g., the upper portion thereof). When assembled for use, the drip chamber may be provided in fluid communication with an IV bag such as by inserting a spike extending from the cap or located at the distal end of flexible tubing extending from the cap into the fluid port of the bag. A priming apparatus (e.g., a priming valve) for priming the drip chamber may be provided in the fluid path upstream of the outlet of the drip chamber. The priming apparatus includes an outflow (or vent) passage and a means for selectively (i.e., by operation of the user) opening and sealing the outflow passage. In some embodiments, the priming apparatus is integrated with the drip chamber cap. In some embodiments, the priming apparatus is integrated into a drip chamber cap having an integral spike. In other embodiments, a spike is separately formed from the drip chamber cap that includes the priming apparatus and the spike is connected to the drip chamber cap, either directly or via intermediate tubing, when assembling of the IV tube set for use. In some embodiments, a modular system may be provided that includes a cap with a priming apparatus incorporated in the cap, which is interchangeably usable with multiple different inflow members (e.g., one or more spike(s) and/or one or tubing members of different diameters), with multiple filter components that provide different drip rate and even with drip chambers of different configurations.

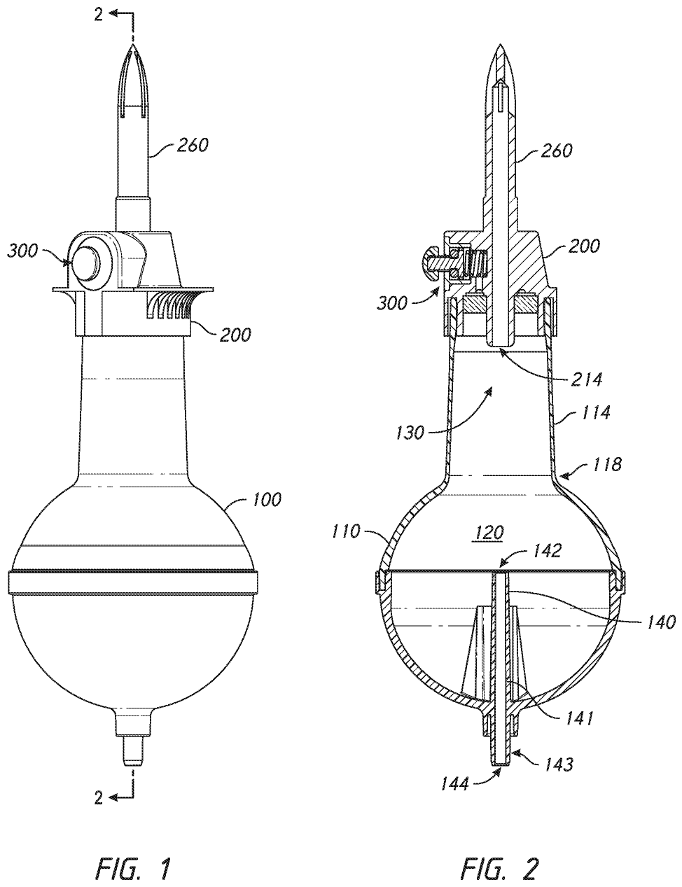

FIGS. 1 and 2 show views of an exemplary emboli-reducing drip chamber with a cap in accordance with some embodiments of the present disclosure attached to the drip chamber. The drip chamber 100 includes a substantially spherical drip chamber body 110 that defines a drip chamber volume 120. Fluid is introduced into the drip chamber body 110 through a drip chamber inlet 130 and the fluid exits the drip chamber 100 through a drip chamber outlet 140. The drip chamber outlet 140 may be provided by an elongate structure (e.g., an outlet tube 141) that extends into the interior of the drip chamber body 110. The drip chamber outlet 140 may be positioned within the drip chamber volume 120 such that its distal opening 142, which may be arranged substantially centrally within the drip chamber volume 120, remains submerged in the fluid irrespective of an orientation of the drip chamber 100 when the drip chamber 100 is filled with a predetermined amount of fluid (for example filling at least 50%, 60% or more of the spherical body). The predetermined amount of fluid may be indicated on the drip chamber 100, in some examples, by a fill line. By maintaining the distal opening 142 submerged under the fluid in the drip chamber 100, the risk of gas entering the tubing downstream of the drip chamber outlet 140 is substantially eliminated, irrespective of the orientation in which the drip chamber 100 is positioned during use, which in turn reduces the risk to the patient of air embolism. As such, the present arrangement may particularly valuable when there is no room, time, or personnel to hold the IV bag at the standard elevated position over the patient. A proximal opening 144 of the outlet 140 may be located at the opposite end 143 of the outlet tube 141. The proximal opening 144 is configured for coupling flexible tubing thereto to provide an IV line to the subject (e.g., human or non-human patient). For example, the end 143 of the structure 141 may be provided with a luer connector for forming a leak-free connection between the drip chamber and the flexible tubing. While shown as spherical, in other examples the emboli-reducing drip chamber body 110 may have a different geometry such as oblong, polygonal or different three dimensional shape, as long as the distal opening 142 of the drip chamber outlet 140 is located at substantially the geometric center of the drip chamber body 110 to enable, in use, the continuous immersion of the distal outlet 142 within the fluid. In yet other examples, a cap with a priming apparatus according to the present disclosure may be used with a conventional drip camber that lacks the emboli-reducing features of drip chamber 100. For example a, cap with priming apparatus may seal the inlet of a drip chamber designed for conventional use at substantially vertical orientation and an elevated position above the patient or subject.

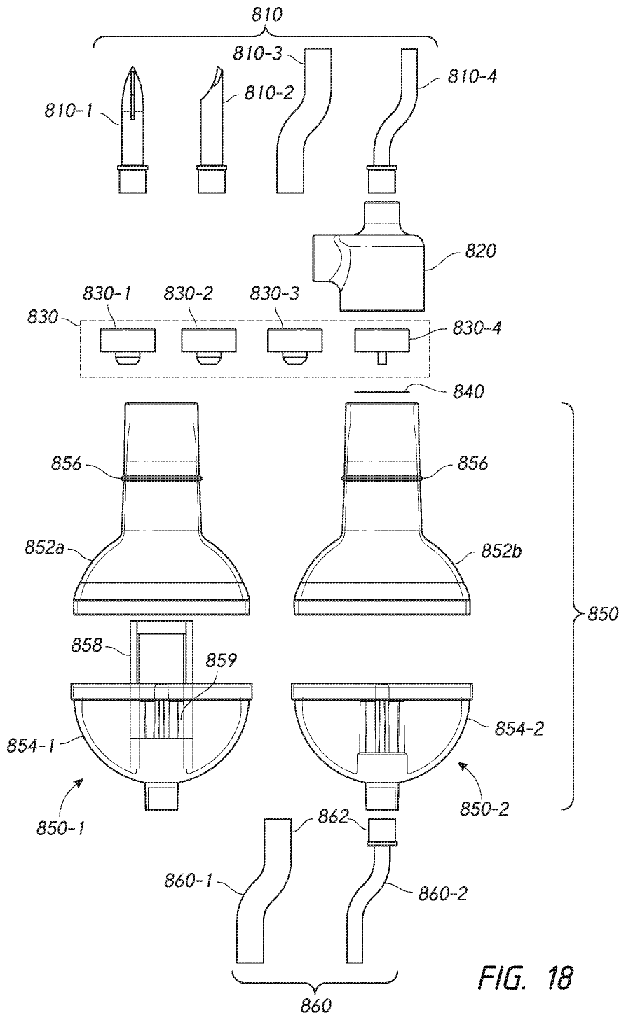

In some embodiments, the drip chamber 100 may be provided with a fill level indicator (see e.g., fill line 856 in FIG. 18), which may be embossed or provided by other suitable raised structure or it may be printed (e.g., overmolded, laminated, screen or laser printed, etc.) on the spherical body 110, to indicate to the user the fluid amount that should be provided in the drip chamber 100 for proper operation of the emboli-reducing function of the drip chamber 100. In some embodiments, the proper fill level may be indicated differently. For example, the drip chamber 100 may include a neck portion 114 extending from the spherical drip chamber body 110 and which defines, at its distal end, the inlet 130 of the drip chamber. The neck portion 114 may thus provide the generally spherical drip chamber with an elongate observation portion or window, in this case just downstream of the drip chamber inlet 130, for monitoring the drip rate. The neck portion 114 may have a substantially cylindrical or slightly tapered or frustoconical shape. In some examples, the interface 118 between the neck portion 114 and the spherical body 110 (also referred to as neck line 118) may function as the fill level indicator. That is, the relative sizes of the spherical body 110, neck portion 114, and elongate structure 141 may be selected such that submersion of the distal opening 142 is ensured when the drip chamber is filled with fluid substantially up to the neck line 118. In some embodiments, the fluid amount that ensures proper operation of the drip chamber (or submersion of the opening 142 in fluid at all orientations) may thus be an amount sufficient to substantially fill the spherical body up to the neck line, referred to here as the drip chamber volume. In some embodiments, the fluid amount for proper operation may be an amount less than the full volume, such as at least 60% of the full volume, at least 70% of the full volume, or at least 90% of the full volume. The size (e.g., length, width, and thus additional internal volume) of the neck portion may be selected in combination with the proper fill level, such that the opening 142 of outlet 140, which in the illustrated example is approximately at the geometric center (or midpoint) of the sphere, remains submerged in the fluid regardless of the orientation of the drip chamber when filled with the predetermined amount of fluid while still providing a sufficiently long observation window for monitoring or confirmation of a desired drip rate. In some embodiments, the fill line may be located in the neck portion of the drip chamber 100.

In some embodiments, the drip chamber 100 may be made from two separately formed parts that are joined together to form the drip chamber. For example, for a substantially spherical drip chamber 100, an upper portion may include the top substantially half spherical portion of the spherical drip chamber and the optional a neck portion, and a lower portion may include the bottom substantially half spherical portion of the spherical drip chamber, which includes the outlet 140. Forming the drip chamber from multiple parts may provide one or more advantages, such as facilitating ease of manufacture, enabling the inclusion of internal components (e.g., a blood filter) and/or enabling the different parts of the drip chamber (e.g., the upper and lower halves) to be made from dissimilar materials. For example the lower portion of the drip chamber may be formed of a relatively rigid material and the upper portion may be formed of a relatively more flexible material at the top. For medical applications, a suitable flexible material may be, but is not limited to, medical grade flexible PVC, or soft durometer polyurethane. A flexible thermoplastic or other flexible material suitable for the desired (e.g., medical, veterinary, sports) application may be used. Suitable rigid materials for forming the drip chamber or a portion thereof (e.g., the bottom portion) may include medical grade acrylic, polyurethane, other suitable hard plastics, glass or metal. In some embodiments, both the upper and lower portions of the drip chamber may be made from a substantially rigid material (e.g., medical grade acrylic or any other suitable hard plastic, glass or metal). In yet further examples, the drip chamber may be formed as a single, unitary component, for example through an additive manufacturing (3D printing) technique.

The drip chamber (e.g., either as a unitary component or in parts) may be manufactured using any suitable technique such as molding, casting, additive manufacturing, machining, etc. These processes may include, but are not limited to, injection molding, polyurethane casting, silicone molding, or Soft Cast TPU (thermoplastic polyurethane) methods. In some embodiments, the drip chamber, or a portion thereof (e.g., the bottom portion) may be made from a material other than plastic, for example a metal. In some embodiments, at least a portion of the drip chamber 100, e.g., the neck portion 114 may be made from sufficiently clear material (e.g., clear plastic) to be usable as a monitoring window, e.g., to view the drip rate. In other embodiments, the drip chamber may be made from two or more parts of the same material or material with similar properties (e.g., rigidity). In yet other embodiments, the drip chamber 100 may be made as a unitary body, from the same or a combination of suitable materials (e.g., via injection molding, overmolding, additive manufacturing or combinations thereof). In conventional systems, making the drip chamber, or at least a portion thereof, flexible may have been a necessity to facilitate priming, e.g., by pumping, the drip chamber in order to fill the drip chamber to an appropriate fill level. A priming apparatus (e.g., priming valve 300) according to the present disclosure obviates the need for manually pumping to prime the drip chamber, and thus the drip chamber may now be entirely made from a suitable rigid material (e.g., a rigid plastic), which may provide for a more rugged design that may be more suitable for use outside of a hospital setting.

As shown in FIGS. 1-3, a cap 200 covers the inlet 130 of the drip chamber 100. The cap 200 defines a fluid passage 220, which communicates fluid from the IV bag into the drip chamber 100. The fluid passage 220 has a distal end and a proximal end. The proximal end of the fluid passage 220 may terminate at an outlet tube 240, which may extend into the drip chamber inlet 130 when the cap 200 is operatively coupled to the drip chamber 100. The outlet tube 240 may be configured for dripping the IV fluid into the drip chamber 100 at a desired rate. For example, the internal diameter of the outlet tube 240 may be selected to provide the desired drip rate. The internal diameter of the outlet tube 240 may be significantly smaller than, for example, the drip chamber inlet 130 to cause the fluid to pass into the drip chamber in distinct droplets and thereby enable counting for determining the drip rate. In some embodiments, the drip rate of the fluid transmitted through the cap 200 may be adjusted or modified by a drip rate adjustor extending proximally from the fluid passage 220.

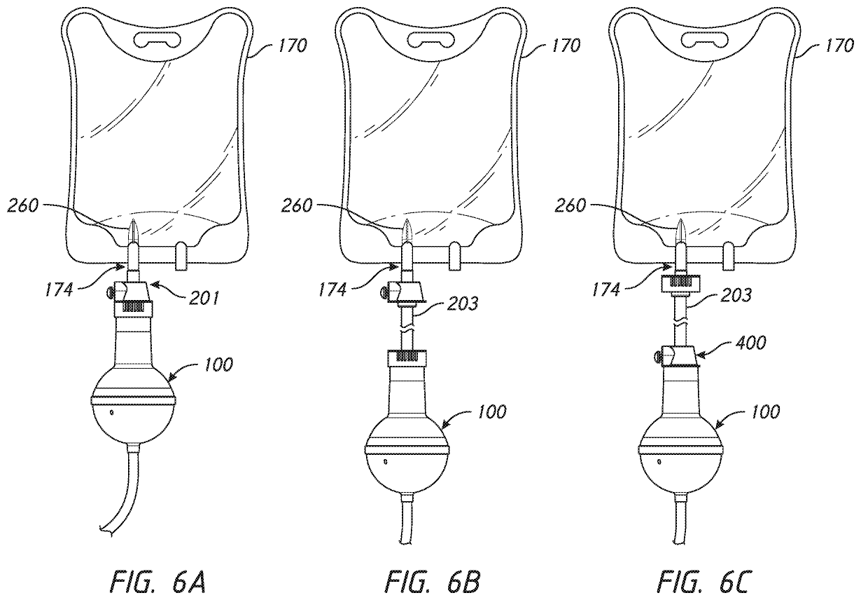

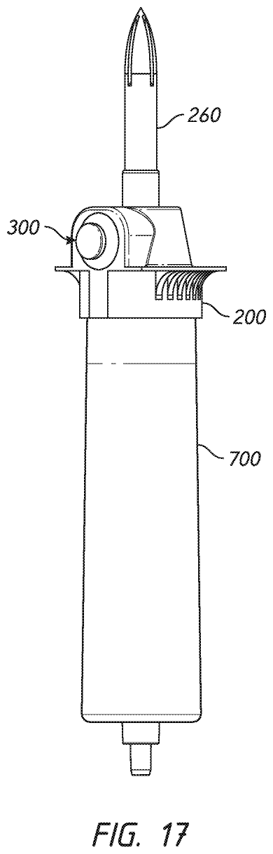

The cap 200 incorporates a priming apparatus according to the present disclosure. The priming apparatus may be implemented by a priming valve 300 integrated into the cap 200 and operable to prime the drip chamber 100. The priming apparatus (e.g., valve 300) may additionally be usable for substantially purging all excess air from the fluid system, such as substantially all the air contained in the IV bag before the bag is connected to the subject, as will be described further below. In this example, the cap 200 is a unitary component (e.g., integrally formed) with a spike 260 that is used to connect the drip chamber 100 to the source of fluid (e.g., an IV bag). In other embodiments, the priming apparatus (e.g., valve 200) may be integrated into the cap or the spike, which are separable from one another by flexible tubing 203 (e.g., as shown in FIGS. 6B, 6C, 7 and 8). In some embodiments, the priming apparatus may be integrated into a cap which is interchangeably connectable to any one of a plurality of different inflow member (e.g., a spike or tubing) as described further with reference to FIG. 18. The priming apparatus (e.g., valve 300) described in this example with reference to a substantially spherical emboli-reducing drip chamber 100, the valve 300 and/or a drip chamber cap (e.g., with or without a spike) that incorporates the valve 300 may be usable with various other drip chambers, some of which may have non-spherical shape (e.g., drip chamber 700 illustrated in FIG. 17) and some of which may not be provided with emboli-reducing functionality.

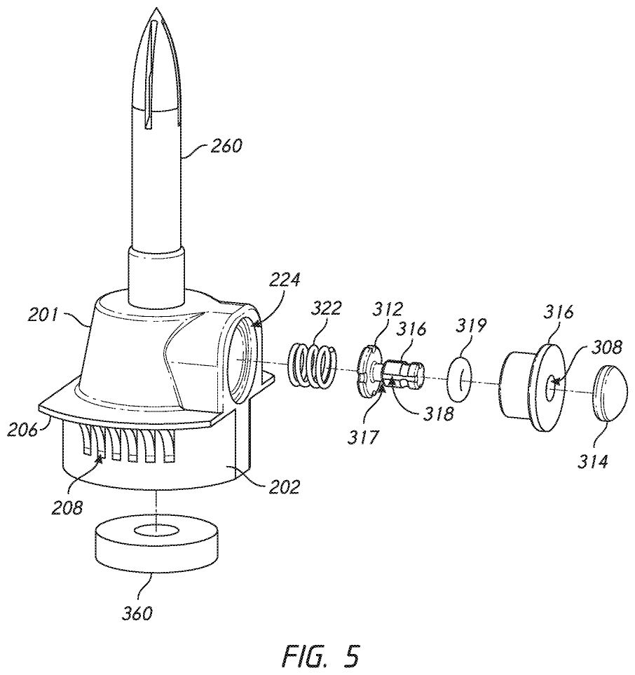

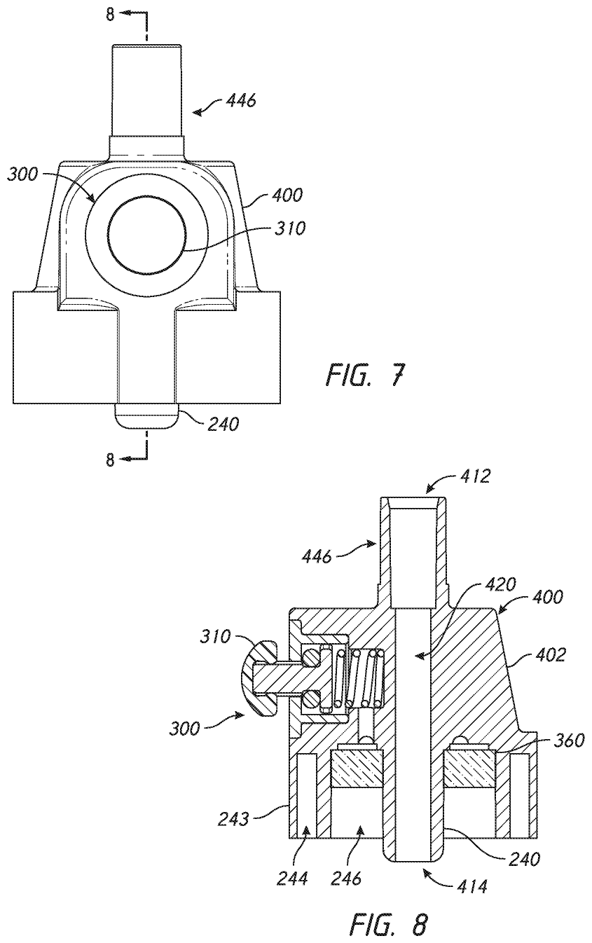

With reference to FIGS. 4 and 5, the cap 200 includes a cap body 202. The cap body 202 may be formed as a unitary component (e.g., a molded or 3D printed body) of any suitable material such as plastic. The cap body 202 is configured to cover the inlet of the drip chamber 100. In some embodiments, the cap body 202 may be separately formed form the drip chamber 100 and subsequently suitably attached to the drip chamber 100 to cover the drip chamber inlet. In other embodiments, the cap body 202 is integrally formed with at least a portion of the drip chamber, for example the upper portion of a multi-part drip chamber 100. The cap body 202 may have a frustoconical shape, as in the present example, or a substantially cylindrical shape (e.g., as shown in FIGS. 18-20). In other examples, the cap body 202 may have a different suitable shape. The cap body 202 has a distal opening 212 (also referred to as inlet 212), a first proximal opening 214 (also referred to as outlet 214), and a fluid passage (or simply passage) 220 connecting the inlet 212 to the outlet 214 of the cap 200 for delivering fluid from the IV fluid container (e.g., IV bag 170 in FIG. 7A) through the cap 200 into the drip chamber 100. The first proximal opening (or outlet) 214 is open on the proximal side of the cap 200 such that when the cap 200 is operatively connected to the drip chamber 100, the outlet 214 faces and is in communication with the interior of the drip chamber 100. In this example, the spike 260 is integral with the cap 200 and thus the cap 200 may also be referred to as an integrated spike cap 201. In other examples, the spike for connecting the drip chamber to the IV bag may be separable form the cap, as in the examples in FIGS. 7-8 and 18-19. In the example in FIGS. 4 and 5, because the spike 260 is integrated with the cap body 202, the inlet 212 to the fluid passage 220 is also the inlet of the spike 260 located near the pointed end 262 of the spike 260. The outlet 214 of the fluid passage 220 is provided at the opposite, proximal side of the spike cap 201, and more specifically here at the proximal end of the outlet tube 240. The cap 200 defines a single fluid path from the IV bag into the drip chamber 100. This single fluid path may be provided by a single fluid passage 220 as illustrated or by a plurality of fluid passages that connect the inlet 212 to the outlet 214. The spike 260 of cap 200 can thus be described as a non-vented spike, in contrast to vented spikes that provide an additional vent passage through the spike, separate from the fluid passage, to permit ambient air to pass into the IV fluid container to displace the volume of IV fluid drawn from the IV container, which particularly with rigid containers can prevent the formation of vacuum that may interfere with the proper flow rate of fluid out of the container.

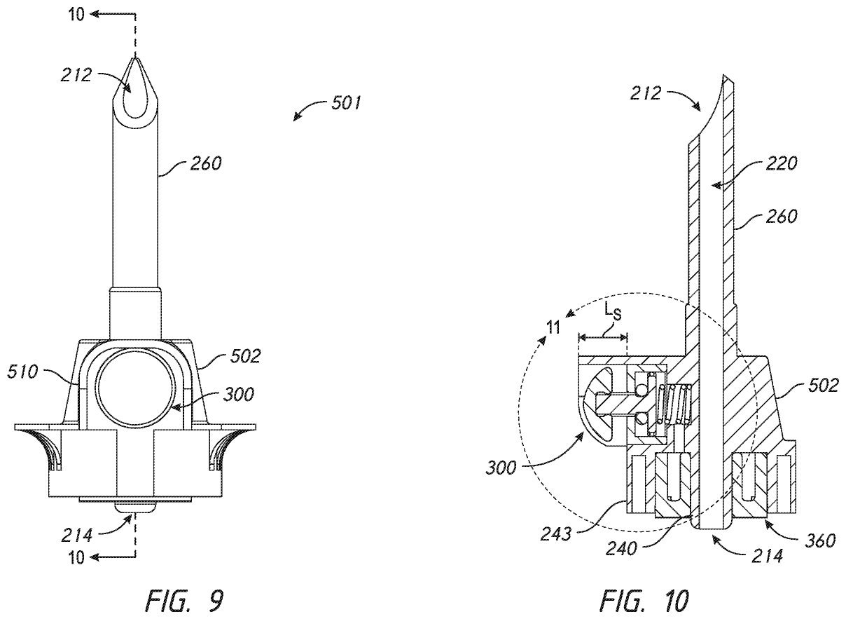

The spike 260 may be configured to promote the flow of fluid. Depending on the type of IV fluid, the configuration of the spike 260 may vary. As shown e.g., in FIG. 4, the inlet 212 at the pointed end 262 of the spike 260 may be provided as one or more through slots 264 located near the tip of the spike 260. Each slot 264 may define a through opening from the outer surface of the spike 260 to the internal fluid passage 220 and may extending substantially longitudinally along the length of the spike (e.g., from the tip of the spike toward the cap body 200), which provides an increased length of the aperture of the inlet 212 to promote or increase the flow of fluid from the IV bag into the fluid passage 220. In another example, the pointed end of the spike 260, and thus the elongated flow promoting inlet, may be provided by a bevel at the distal end of the spike, such as in the examples in FIGS. 9-13. This configuration may provide a larger sized opening into the fluid passage 220 that may be suitable for use with certain fluids (e.g., colloids) while still providing and elongated flow promoting inlet to generally promote the flow of fluid into the passage 220. Prior to use, the spike 260 may be covered by a sleeve to keep the spike sterile, and which may be fitted and retained to the spike via a spike shoulder 268 provide at the base of the spike 260. In use, after removal of the sleeve, the spike may be inserted into the fluid port 174 of a bag 170 (see e.g., FIG. 6A) and in some cases the shoulder 268 may also function as a hard stop that limits insertion of the spike into the fluid port 174.

The cap body 202 is configured to be coupled to the neck portion of the drip chamber to cover the drip chamber inlet 130 whereby the outlet 214 of the cap 200 is positioned facing, and in some cases inside, the drip chamber 100, as shown in FIG. 2. The cap 200 may include a joint interface located on the proximal side of the cap body for coupling the cap to the drip chamber. For example, the cap body may define an annular groove that provides the female portion 243 of a male/female coupling 245. The male/female coupling 245 is implemented here as an annular tongue and groove joint. The male/female coupling 245 of the present example includes an annular groove 244 provided on the cap body 202, in this case extending along the perimeter of the underside of the cap body 202 and facing the drip chamber, that functions as the female portion 243 of the male/female coupling 245. The annular groove 244 is configured to receive the rim 249 of the neck portion 114 to form the annular tongue and groove joint. In this manner, the rim 249 functions as the male portion 247 of the male/female coupling 245. In some embodiments, the male portion 247 (e.g., rim of the neck portion 114) and the female portion 243 (e.g., annular groove 244) may be sized for an interference fit. For example, the internal diameter of the neck portion at the rim 249 may be slightly smaller than the diameter defined by the groove 244 such that insertion of the rim 249 into the groove 244 may be achieved by slightly stretching the opening of the neck portion. The natural tendency of the component (e.g., drip chamber 100) toward its nominal unstretched state may enhance the friction and thus retention or coupling between the drip chamber 100 and the cap 200. In some examples, the rim of the neck portion and the annular groove may be sized for a snap fit whereby the rim portion is slightly deflected from its nominal (as manufactured) state during the insertion into the groove and/or includes snap features, which mechanically engage or interlock with features on the cap to couple the cap to the drip chamber. Any features designed to mechanically interfere with the subsequent separation of the parts may be used as the interlocking or snap features. The male/female coupling 243, among other components of the IV tube set, may be configured to withstand separation at internal pressure of up to 5 psi, and in some cases greater. In some embodiments, the coupling between the drip chamber (e.g., neck portion 114) and cap 200 may additionally or alternatively be achieved via other suitable means, such as by bonding or gluing the two parts together (e.g., via a chemical adhesive, or using laser or RF welding, or the like). A similar tongue and groove joint (or male/female coupling) may be used to join an upper portion of the drip chamber to a lower portion of the drip chamber (see e.g., FIG. 2), in the case of a two-part design. In yet other embodiments, a different type of joint, such as a lap joint or other suitable joint, may be used in some cases in combination with adhesion and/or an additional mechanical fastening means (e.g., snap and/or interlocking features). Any two separately manufactured components, which are to be assembled for use in the tube set, may be attached using various methods including, but not limited to fusing or bonding using solvent bond, ultraviolet (UV) activated glue, sonic welding, over molding, spin welding, and chemical bonding. In cases where parts of dissimilar material are joined, any suitable technique for boding or gluing may be used, such as via UV light cured bonding, overmolding, RF welding, laser welding, etc. However, as described, in some embodiments the drip chamber body and integral neck portion may be a unitary component rather than a multi-part assembly.

Referring to FIG. 5, the cap body 200 may include a flange 206 that extends radially outward from the cap body 202. The flange 206 is configured to provide a surface for pressing the spike 260 upward when inserting the spike into the fluid bag. The flange 206 may be provided by a substantially continuous annular flange extending radially outward from the cap body, or by multiple flange portions, shown here as first (or left) and second (or right) flange portions extending transverse to the spike, in diametrically opposite directions. The flange 260 may be equipped with a traction feature 208, shown here as a plurality of ribs, provided on the underside of each of the flange portions. The traction feature 208 may enhance the ergonomics of the underside of the flange 206 (e.g., for improved fit with the user's fingers), and may additionally optionally structurally re-enforce the flange 206. Any of the drip chamber caps described herein (e.g., cap 400, 900) may include such a flange.

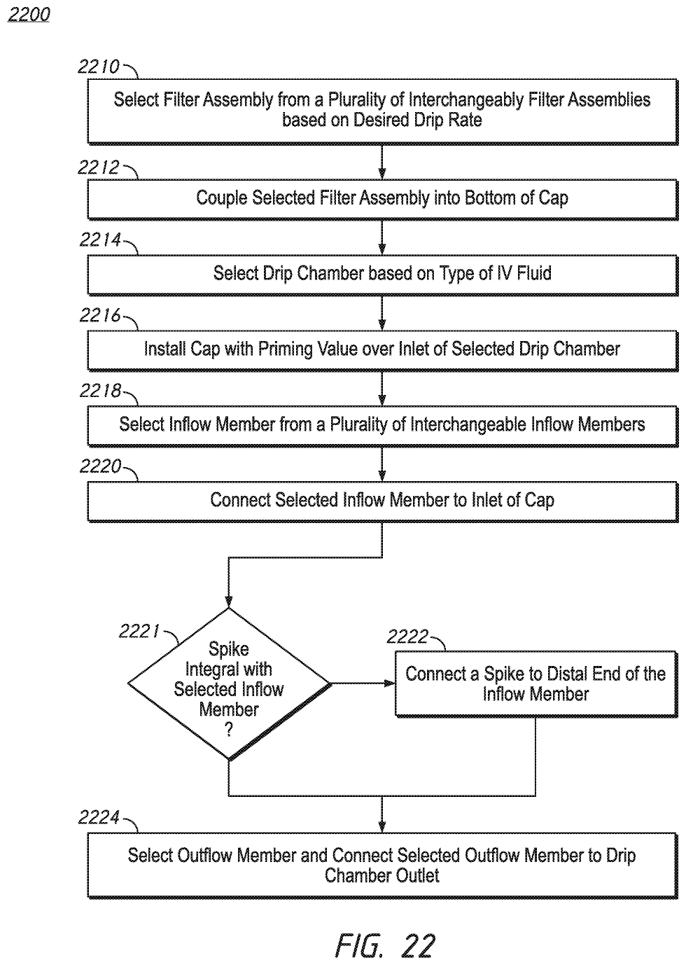

In accordance with the principles of the present disclosure, an IV tube set may be provided with an apparatus that enables priming the drip chamber without “pumping” the drip chamber. Pumping, as is conventionally used for priming the drip chamber, may introduce additional air into the fluid system, which may increase the risk for air embolism especially during rapid infusion with an IV bag subjected to external pressure (e.g., from a compression sleeve) or when the vertical arrangement of the fluid system (e.g., vertical orientation of the drip chamber) cannot be guaranteed. Conventional priming that uses pumping of the drip chamber pushes air from the drip chamber into the IV bag in order to establish the operating fluid height. In contrast, the arrangement described herein may enable the user to purge substantially all unnecessary or excess air from the system during the priming process before infusion begins, as in the example described below with reference to FIG. 22, thus potentially further reducing the risk of the accidental introduction of air bubbles into the patient's blood stream. The tube set described herein may be configured to hermetically seal and maintain internal pressure of up to 5 psi or greater. The joints, which in some examples may include glue joints, between components of the tube set may be configured to withstand the above noted pressures and in some examples to withstand tensile load of at least 15N applied longitudinally along the tube set. In addition to the ability to purge more air out of the system, the priming apparatus described herein also enables the drip chamber to be filled or primed in a fraction of the time (e.g., 1 to 2 seconds) as compared to priming via conventional “pumping” which may take 10 or longer.

As shown in FIGS. 2-5, a priming valve 300 may be provided in a cavity 204 of the cap body 202. An inlet opening 306 connects the cavity 204 to the drip chamber allowing air to pass from the drip chamber into the cavity 204. An outlet opening 308 connects the cavity 204 to the exterior of the system (i.e. to the ambient air) allowing the air to exit out of the cavity 204 into the exterior. The valve 300 includes a closure mechanism (e.g., actuator) is operatively arranged along the fluid path defined from the IV bag to the drip chamber to selectively communicatively couple the fluid passage 220 to the exterior and thus to ambient pressure. In some embodiments, the valve 300 is configured to achieve this function without compromising the sterility of the fluid path. For example, the valve 300 may include a barrier 360 (e.g., filter or porous membrane) configured for maintaining sterility, for example by preventing the passage of bacteria or other microbes through the barrier 360. The barrier 360 (e.g., filter or porous membrane) may have an appropriate micron rating, for example in the range of 0.2 microns to about 1.4 microns. The filter may have any suitable pore size (or micron rating) that sufficiently small to block the passage of bacteria or other microorganism as may be desired for a particular application. Additionally or alternatively, the barrier 360 may be configured to repel liquids (e.g., the barrier may be aquaphobic) such as by including or being treated with an aquaphobic material to improve the valve ability to resist the passage of water therethrough. The barrier 360 may be provided at any suitable location across the inlet opening 306 to the valve cavity 204. The physical parameters of the barrier 360, such as micron rating, thickness, type of material, etc. may be further selected or varied to tailor other performance aspects of the barrier 360 such as the rate of air flow through it, for example for tailoring the speed at which the valve 300 is able to evacuate the air from the fluid path.

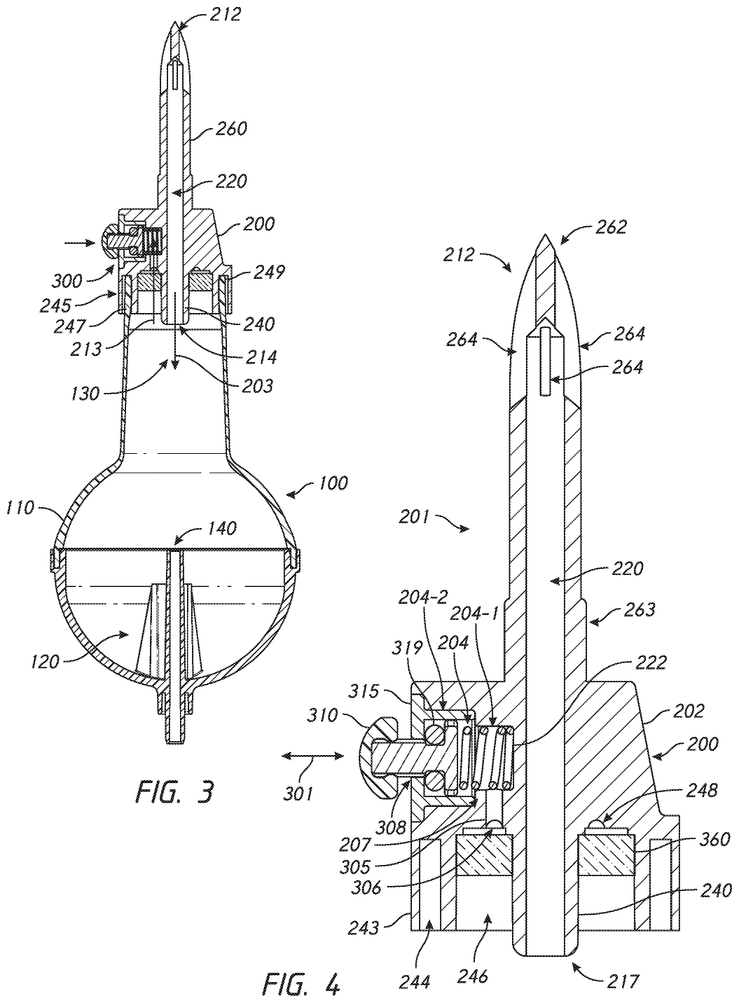

The outlet opening 308 can be selectively opened and closed using an actuator (e.g., button 310). In this example, the actuator moves along the direction indicated by arrow 301 to break the seal between sealing element (e.g., o-ring 319) and the outlet opening 308 to selectively open and close the valve. When in the open position, the cavity 204 is open to the exterior (i.e. to ambient air) thus allowing the air to vent out of the drip chamber through the cavity 204 (as shown by arrow 213 in FIG. 3). When the valve is closed, such as after priming the drip chamber, the outlet 308 of the cavity 204, and thus the cavity 204, is hermetically sealed from the ambient air, preventing air from passing into or out of the cavity and thus into or out of the fluid system (e.g., the interior of the drip chamber, cap, upstream IV line and/or IV bag). The cavity 204, thus, provides a passage to selectively communicatively couple the drip chamber interior to the exterior (i.e. ambient air) which can facilitate the purging of air out of the system. When properly operated to prime the system, opening the valve 300 causes the air in the IV bag to pass into the drip chamber through the fluid passage. For example, when operated with a pressure cuff, when the valve is open and the interior of the fluid system is connected to the ambient pressure, the lower ambient pressure causes the flow of air/fluids out of the bag and into the drip chamber, as shown by arrow 203. At the same time, air is purged from the drip chamber through the valve as fluid fills the drip chamber. The valve is maintained opened until a desired amount of fluid (e.g., a fluid up to the fill line) is dispensed form the bag into the drip chamber and the valve is then closed. The inlet opening 306 to the cavity 204 may be formed on the proximal side of the cap body such that when the cap 200 is fixed to the drip chamber 100 to cover the inlet 130 of the drip chamber, the inlet opening 306 is in fluid communication with the interior of the drip chamber. In other examples, the inlet opening to the valve cavity may be suitably positioned elsewhere. In some examples, a porous barrier 360 (e.g. any suitable porous membrane or filter having appropriate micron rating to reduces or substantially prevent the passage of microbes through it) may be provided across the inlet opening 306.

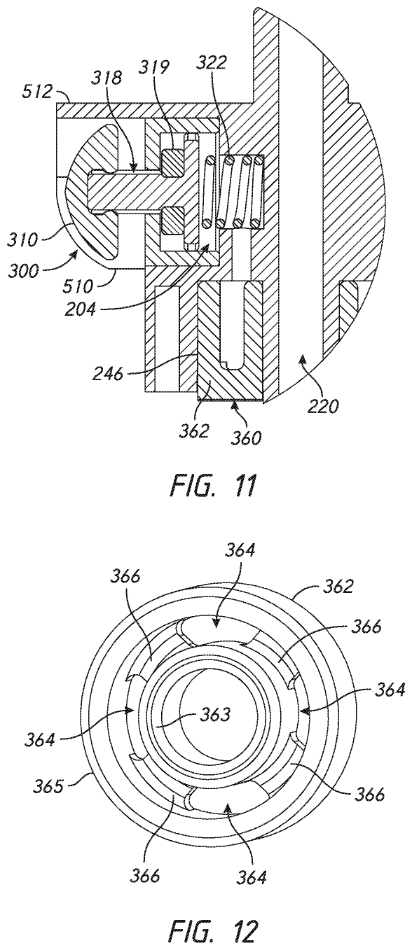

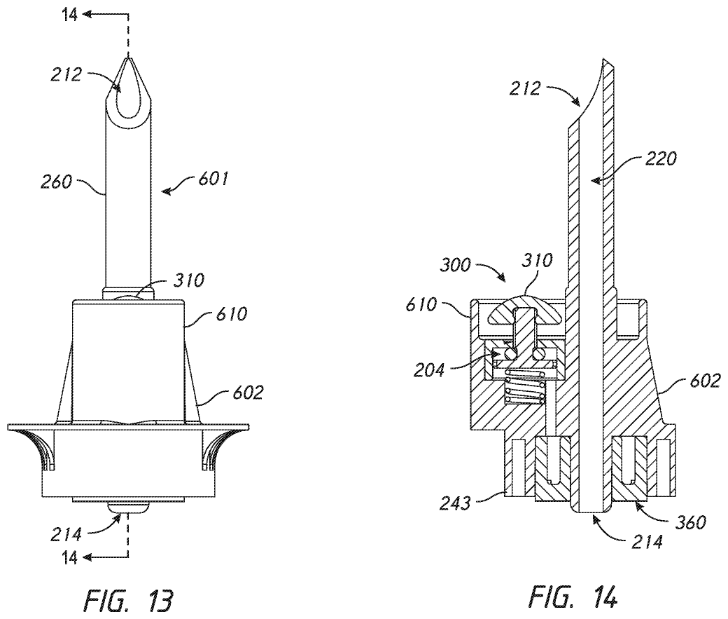

The closure mechanism can be implemented using any suitable mechanism for selectively opening and sealing the outlet opening 308 of the valve. The closure mechanism may include an actuator, implemented in the present example by a button 310 configured to be depressed to open the valve and released to seal the valve. Referring to FIGS. 4 and 5, the button 310 has a substantially circular base 312 received within the substantially cylindrical cavity 204 and movable along the direction 301. The cavity 204 is define by a first wall 222 opposite the mouth 224 of the cavity 204, and side wall(s) 226 extending from the first wall 222 to the mouth 224 of the cavity 204. The button 310 includes an actuation end 340, which is configured to remain outside of the cavity 204 when the valve is in either the open position or the closed position. The actuation end 340 may be positioned at any suitable location for actuation, such as along a peripherally-facing side (e.g., as in the examples in FIGS. 1 and 9), or a distally-facing side of the cap body 202 (e.g., as in the example in FIG. 13). The base 312 and the actuation end (e.g., shown here as button cap 314) of the button 310 may be connected by a post 316 that passes through the outlet opening 308. The post 316 may be sized to move freely within the opening 308 and thus allow for the passage of air through the valve 300. Optionally, the post 316 may be fluted, shown here as having one or more channels 318 along the length of the post, to increase the outflow of air when the valve is opened. The closure mechanism of valve 300 may also include a seal, shown here as an O-ring 319, which is positioned to bear against an inner side of the wall that defines the outlet opening, in this case the inner side of the plug 315, to seal the outlet opening 308 when the valve is in the closed position. As shown, the base 312 has a dimension (e.g., diameter) which is greater than a corresponding dimension (e.g., diameter) of the outlet opening 308 such that, when assembled, the seal (e.g., O-ring 319) is sandwiched between the base 312 and the inner side of the plug 315. The seal may have a generally circular transverse cross section (e.g., as shown in FIG. 4) or the seal may be a flat seal, having a different transverse cross-section (e.g., an O-ring with rectangular cross-section, also referred to as flat O-ring, as shown in FIG. 11) or may have yet another suitable form factor.

The button 310 may be biased toward the closed position. For example, a biasing element, shown here as a helical spring 322, is operatively positioned with respect to the button 310 to force the button 310 toward the position in which the valve 300 is closed, which in this case is a position in which the button 310 extends further out from the cavity 204 than in the open position. In the present example, the spring 322 is positioned between the first wall 222 of the cavity 204 and the base 312 of the button 310, thereby urging the button 310 in a direction out of the cavity 204. However, in other examples, a different operative arrangement that biases the actuator of the valve toward the closed position may be used. The seal 319, which may be made from any suitable material such as an elastomer (e.g., rubber or silicon), any suitable thermoplastic or thermoset material (e.g., a polyurethane) or other, may be retained to the button 310 such that the seal moves with the button 310, as the button is moved between the closed and open positions. The seal may be retained to the button using any suitable means such as an adhesive or mechanical means, for example by being seated in an annular groove 317, e.g., at the base of the post 316. In use, assuming no mechanical lock is engaged, the user can apply sufficient force (e.g., user force F) to overcome the biasing force of the spring to freely actuate the button 310 between the open and closed positions any number of times as may be desired.

For ease of manufacturing, the mouth 224 of the cavity 204 may be sized to accommodate passage of the base 312 therethrough. Once the base 312 has been inserted into the cavity 204, the mouth 224 may be covered by a plug 315, for example by press-fitting the plug 315 into, and optionally gluing the plug to the body 202. The plug 315 defines an aperture that provides the outlet opening 308. During assembly, the plug 315 is sleeved over the post 316 such that the post 316 passes through the outlet opening 308, and a button cap 314 may then be attached to the free end of the post to provide the actuation end of the button. The button cap 314 may be attached to the post via any suitable means, including but not limited to a snap fit, a press fit, a mechanical fastener, or glue. Similarly, for ease of manufacture, the button 310 may be formed of separable components to facilitate installation of the plug 315, or alternatively, the plug 315 may be formed as two halves to facilitate installation around the post 316 of the button 310, which may in the latter instance be formed as an integral or unitary component.

As shown in FIG. 4, the cavity 204 may include a ledge 207, which divides the cavity 204 into an inner portion 204-1 and an outer portion 204-2, in this case the outer portion having a larger diameter than the inner portion. The outer portion 204-2 is sized to accommodate the base 312 of the button 310, allowing the button 310 to reciprocate along direction 301 within the upper portion 304-2. The inner portion 204-1 is sized to accommodate the helical spring but does not permit passage of the button 310 into the inner portion 204-1. As such, the ledge 207 functions as a hard stop to limit the movement of the button 310. When assembled, the spring 322 is received in the inner portion 204-1 and rests against the first wall 222 of the cavity 204. The ledge, acting here as a hard stop, thus also limits the amount of compression of the spring 322 to reduce the valve actuator.

As previously described, the proximal end of the spike cap 201 may be configured for securely coupling the spike cap 201 to the neck portion 114. In addition, the proximal end of the spike cap 201 may be configured to support a barrier 360, shown here as filter 360. The filter 360 may be secured to the proximal side of the cap such that it is positioned inside the drip chamber between the inlet to the valve (i.e., inlet opening 306) and the outlet of the spike (i.e., outlet 214). In some examples, the filter 360 may have an annular shape configured to fit (in some cases, in an interference fit) within an annular bore 246 provided on the underside of the cap 200. The annular bore 246 may be defined between the outlet tube 240, which may extend generally centrally from the underside of the cap body 202 and the inner walls of the female member of the male/female coupling 243. In some embodiments, the filter 360 may additionally be glued or bonded to the cap 200, or to an intermediate component (e.g., filter retainer 362 in FIG. 11 or other suitable support structure), which may be inserted (e.g., press fit) and/or bonded to the cap 200. The barrier (e.g., filter 360) may be antimicrobial, water resistant, or water tight. In some examples, the barrier (e.g., filter 360) may be hydrophobic, e.g., by being formed of or coated with a hydrophobic material. For example, the barrier (e.g., filter 360) may be be or include a polytetrafluoroethylene (PTFE) membrane or other suitable material that resists or repels the passage of water through it. The barrier (e.g., filter 360) may be a multilayer structure in some examples. The filter's porosity or micron rating may be tailored or selected to block or strain microbes of any desired size and thus type and may thus aid in maintaining the sterility of the tube set in use.

As previously described, the valve cavity 204 may have its inlet opening 306 located on the underside of the cap body, as in the present example, the inlet opening 306 being connected to the cavity 204 via a passage 305 that extends from the annular bore 246 to the cavity 204. The passage 305 may be substantially parallel to the fluid passage 220, as shown in FIG. 4, or may have other suitable geometry. In some embodiments, an annular channel 248 may be formed at the base of the bore that connects with the passage 305. The annular channel 248 may lie behind the filter 360 (or the support structure carrying filter 360) providing an air space behind the filter (or filter/support combination) for more efficient channeling the outflow of air into the valve 300. In some embodiments, the filter's porosity, the geometry of the channel 248, if present, and/or the geometry of the passage 207 may be tailored to obtain a desired resistance to the outflow of air and thus to the rate of purging of air from the drip chamber during priming. In some embodiments, the valve may be configured to enable the purging of air and consequently the filling of the drip chamber in under 5 seconds, and in some cases within 1 to 2 seconds upon opening of the valve 300. When the valve is closed, the fluid path from the IV bag to the drip chamber volume 120 may be sealed, with the valve configured to maintain a leak proof seal at pressure up to 5 psi or greater.

As noted, in some examples, the priming valve 300 may be housed within the body of an integrated spike cap 201 which is directly coupled to the drip chamber 100, e.g., as shown in FIG. 6A and described in detail above with reference to FIGS. 1-5. In other examples, the priming valve may be located elsewhere along the fluid path such as within the body of a spike 260 which is separated from the drip chamber 100 by flexible tubing 203, e.g., as shown in FIG. 6B. In yet other examples, the priming valve may be housed within the body of a cap 400 that is separated from the spike 260 by flexible tubing 203, as shown in FIG. 6C. Also, while examples of the cap and priming valve assemblies are described here with reference to an air emboli-reducing (or all-position drip chamber), the caps (with spike or without) and priming valve assemblies of the present invention may be used with any other drip chamber geometry, whether convention, air emboli-reducing or not, such as to enable more expedient priming and the reduction of air within the system.

FIGS. 7 and 8 show an example of a cap 400 incorporating a priming apparatus (e.g., valve 300) according to the present disclosure. The cap 400 may have similar features that operate similarly to those described with reference with the integrated spike cap 201, with the primary difference here being that the cap 400 does not have an integrated spike. Instead, the spike which is separate from the cap 400 may be attached to the cap 400 via tubing (not shown in this view). The distal side of the cap 400 may be equipped with a luer fitting 446 or any other suitable means for establishing a leak free connection to the tubing for connecting the cap 400 to the spike and IV bag. Similar to the cap 200, cap 400 includes a cap body 402 having a distal side that defines a distal opening or inlet 412 and a proximal side that defines a proximal opening or outlet 414. The inlet 412 receives fluid from the IV bag and the fluid is transmitted via the fluid passage 420 to the outlet 414 and into the drip chamber positioned to face the proximal side of the cap 400. The fluid passage 420 connects the inlet 412 to the outlet 414 and defines the fluid passage for both delivering fluid from the bag into the drip chamber and for passing the excess air from the bag into the drip chamber at the start of the priming process.

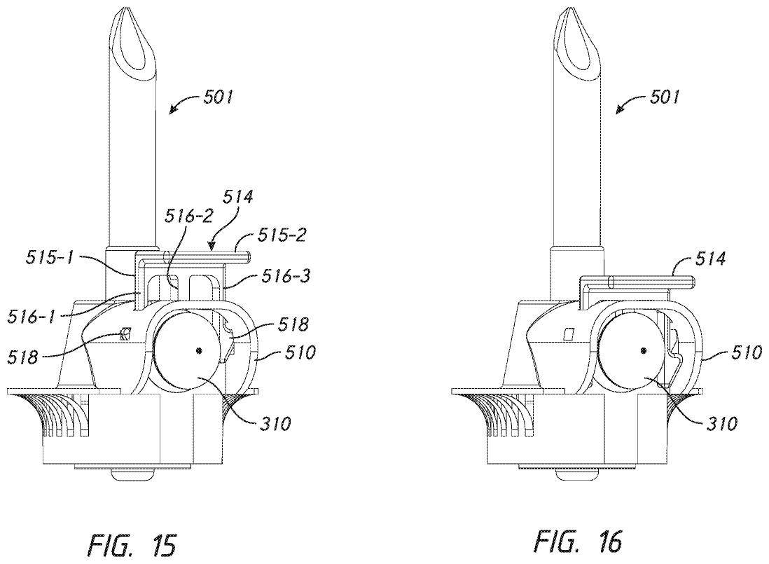

In some examples herein, the actuator (e.g., button 310) for opening and closing the valve 300 extends from a peripheral side of the cap body (e.g., body 202 or body 402). An apparatus with a priming valve (e.g., valve 300) according to the present disclosure may be further configured to reduce the risk of accidental or unintentional actuation, and thus opening, of the valve 300. FIGS. 9-11 show another example of a spike cap 501 with a priming valve 300 according to the present disclosure. Similar to spike cap 201, the spike cap 501 is an integrated cap and spike used to directly couple a drip chamber to an IV fluid source. As such, the spike cap 501 includes a spike 260 extending directly from the drip chamber cap. The spike cap 501 incorporates a priming apparatus, shown here as priming valve 300. However, in other examples, a priming apparatus of different configuration (e.g., priming valve 901) may be used. The priming apparatus (e.g., valve 300) may include an actuator (e.g., button 310) shown here as extending transversely to the spike 260. In some use cases (e.g., in a battlefield or other emergency response scenario, or in a veterinary setting where it may be impractical to immobilize the “patient”), the tube set (e.g., the IV bag and the drip chamber attached thereto) may move or be moved around, placed near or onto the patient, and generally handled with less concern for its precise placement or orientation during the administration of IV fluids, all of which may increase the risk of accidental actuation of the valve. To reduce this risk, the actuator (e.g., button 310) may be at least partially surrounded by a shroud 510 configured to prevent unintentional actuation (e.g., pressing of the button 310). In some embodiments, the valve actuator may additionally or alternatively be equipped with an actuator lock (e.g., button lock 514 in FIGS. 15 and 16).

In the example in FIGS. 9-11, the shroud 510 is shown as a partial guard wall 512, which in this case wraps around the top and both sides of the button 310. In other examples, the guard wall 512 may extend around different portions of the periphery of the button (e.g., wrapping around one side and both the top and bottom, leaving the opposite side substantially open to facilitate access by the user's finger for actuation). Thus, in some examples, the shroud 510 may only partially surround or enclose the button, providing an opening or access for placement of the user's finger when operating the button. In other examples, the shroud may extend substantially fully around the periphery of the button 310, thus substantially fully surrounding button 310, as in the example in FIGS. 12-13. The shroud may extend substantially perpendicularly to the valve housing, to a length Ls equal to or slightly beyond the projecting portion of the button, as shown in FIG. 10. This arrangement may prevent or reduce the risk of accidental or unintentional actuation by forcing a more intentional placement/alignment of the actuating object (e.g., user's finger) with respect to the button 310 (e.g., with the finger aligned with the side opening of the shroud 510) to actuate the button 310. In other examples, the shroud may be differently configured, such as being differently sized, shaped, and/or positioned. For example, and as shown in FIGS. 12-13, the shroud may be sized such that the button 310, when released, projects slightly beyond the shroud. In such cases, the length of the shroud may be slightly less than that of the projecting portion of the button but still long enough to reduce the risk of accidental depression of the button beyond an amount that would overcome the breaking pressure of the valve, which amount may depend on the strength of the spring, the amount of compression in the seal with the valve fully closed, or combination thereof.

In some examples, the risk of unintentional actuation of the valve may be reduced by positioning the actuator on a side or configuring the actuator for actuation in a direction unlikely to be engaged, other than by intention. With further reference to FIGS. 13 and 14, another example of a spike cap 601 is shown, which may include many of the same features of one or more of the caps described herein. Unlike the earlier examples, the valve cavity 204 and button 310 of the spike cap 601 are oriented substantially in line with the spike 260, thus actuation of the valve, in this example requires, an actuation force substantially aligned with the spike, which may be more difficult to occur accidentally. With this arrangement, when connected to an IV bag, the risk of accidental actuation of the button 310 along the lengthwise direction of the spike may be significantly lower than if the button was peripherally positioned on the cap body. Additionally and optionally, the spike cap 601 may include a shroud 610, as shown in FIGS. 13 and 14, which similarly is oriented in line with the spike. As shown here, the shroud 610 may be a full shroud which substantially fully surrounds the button 310, or it may be a partial shroud extending only around a portion of the periphery of button 310 as in the previous example.

In yet further examples, the risk of accidental actuation may be reduced or prevented by a mechanical locking mechanism operatively associated with the actuator. FIGS. 15 and 16 show another example of the spike cap 501, equipped here with a button lock 514. The button lock 514 is shown here as a generally L-shaped structure, having a first portion 515-1 and a second portion 515-2 generally perpendicular to the first portion 515-1. The first portion 515-1 is forked defining a set of tines, which are inserted through a one continuous aperture or a plurality of discrete apertures in the shroud for engagement with the button and shroud. The first portion of the button lock includes a button engagement feature, shown here as the middle tine 516-2, and a locking feature, provided by the pair of outer tines 516-1 and 516-3. The tines 516-1, 516-2, and 516-3 are connected to one another near the interface between the first and second portions, or they may be connected by the second portion. The second portion provides an actuation end of the button. In other examples, the second portion may be omitted and the button lock 514 may be a substantially planar structure. Other suitable geometries may be used. The spike cap 501 may be provided with the button lock in the open (or disengaged) position, as shown in FIG. 15 and the lock may remain in this position until the tube set has been primed and is ready for use on the patient. Once primed (e.g., air purged) from the tube set and/or the tube set has been connected to the patient, the button lock 514 may be actuated (e.g., pressed) to the lock position, as shown in FIG. 16, whereby the button engagement feature (e.g., time 516-2) is positioned between the button cap 314 and the valve housing to interfere with and thus prevent movement of the button into the valve cavity. The locking feature (e.g., outer tines 516-1 and 516-3) of the button lock 514 are configured to retain the button lock 514 in the open (or disengage) position, the locked (or engaged) position, or both. Here, each of the outer tines 516-1 and 516-3 is provided with a tooth or other suitable structure 518 configured to interlock with a cooperating feature on the shroud 510 at one or a plurality of positions. Here, the teeth 518 on the outer tines 516-1 and 516-3 are arranged to face in opposite directions and toward the nearest portion of the wall of the shroud. The shroud may include cooperating indents or apertures arranged to receive the teeth to lock the button 514 into a first position in which the button lock extends from the shroud, corresponding to the open or disengaged position. The shroud and teeth may also be configured to interlock (e.g., with the teeth engaged the bottom edge of the shroud wall) to lock the button 514 into a second position in which the button lock is lowered into the shroud, corresponding to the locked or engaged position. Other suitable button lock arrangements may be used. In yet further embodiments, a separate lock such as lock 514 may not be used but the button may include a locking features such that the button itself is configured for one time actuation, whereby following a single depressing and release of the button, the button automatically locks into the closed valve position.

As described, the valve may, in some embodiments, include a barrier (e.g. filter 360), which allows air to be purged from the system while preventing microbial transfer from the exterior into the tube set, allowing the system to remain sterile. In some embodiments, the barrier may be configured to additionally restrict the transfer of fluids out of the system. Referring back to FIG. 11, the barrier (e.g., filter 360) may be operatively positioned between the valve cavity 204 or its inlet and the fluid passage 220 or its outlet to restrict or prevent the passage of fluids into or out of the drip chamber without substantially inhibiting the flow of gas (e.g., air). In some examples, the filter 360 may be provided by a thin sheet of porous material, e.g., having a thickness in the range of 5-10 mil (or 5 to 10 thousands of an inch), and may, in some such examples, be carried on a support structure, shown here as an annular filter retainer 362. The support structure (e.g., filter retainer 362) may be used to operatively position (e.g., across the valve inlet) and therein retain the filter more easily and securely. Referring also to FIG. 13, the filter retainer 362 may have a complimentary shape as that of the annular bore 246, and may be sized to be press fit into the bore 246. In some examples, the annular bore 246 may be tapered, decreasing in dimension distally, to facilitate a press fit with the retainer 362. The retainer 362 may additionally or alternatively be bonded to the cap (e.g., to the annular bore 246). The retainer 362 and filter 360 assembly, when received in the bore 246, may substantially fill the bore.

The retainer 362 may have any suitable geometry to effectively support and securely couple the filter 360 to the cap body. For example, the retainer 362 may be sized to substantially fill the bore 246 and may include one or a plurality of through passages 364 to allow the flow of air from the proximal side of the retainer, which carries the filter 360, to the distal side of the retainer 362. Additionally or alternatively, the retainer 362 may be further shaped to reduce its overall weight without adversely impacting the rigidity and thus ability of the retainer to firmly couple the filter to the cap body. The retainer 362 may be formed as a monolithic body from any suitable material, e.g., polylactic acid (PLA), medical grade PVC, polyurethane, or other plastic material suitable for medical/sterile applications, using any suitable manufacturing technique, such as injection molding or 3D printing. In the illustrated example, the retainer 362 body includes a pair of coaxially arranged tubular portions (e.g., inner tubular portion 363 and outer tubular portion 365), connected, at their proximal ends, by a plurality of flanges 366, which are spaced apart by and thus define the through passages 364. Any suitable number and arrangement of the flanges 366 may be used, in this example four flanges, which extend radially from the inner to the outer tubular wall in diametrically opposite directions. In other examples, fewer or greater number of flanges may be used, which may be equally spaced or arranged in a different, irregular pattern. The filter 360 may be coupled (e.g., bonded) to the distal side of the retainer to span across the passages 364 such that any air flow through to the valve passes through filter 360.

In use, a tube set according to the present disclosure may offer one or more advantages. In use, as shown for example in FIG. 6A, flexible tubing may be connected to the drip chamber outlet (e.g., to outlet tube 141), this tubing being referred to here as proximal tubing. The proximal tubing is clamped, e.g., with a roller clamp or other device capable of stopping the flow through the proximal tubing. The sleeve is removed from the spike 260 and the spike is inserted into the source of fluid (e.g., IV bag 170). Most IV bags include a sizeable amount of air or other gas within the bag along with the IV fluid. Generally, when infusing the fluid under gravitation pressure, the air in the bag may not pose a significant risk as typically there is significantly more time to stop the IV and replace the IV bag when all of the fluid has been administered. However, when infusing fluid under elevated pressure, air in the IV bag and air added to the bag through pumping during the priming of the drip chamber may increase the risk of air embolism to the patient. Thus, purging the unnecessary air from the fluid system may significantly reduce the risk of embolism in the patient.

After the IV bag has been spiked and the proximal tubing has been clamped, the IV bag may be subjected to elevated pressures, e.g., via a pressure cuff as described in co-pending U.S. Ser. No. 16/093,552 and U.S. Ser. No. 15/537,189, the disclosures of which are incorporated herein by reference. As the IV bag is brought to elevated pressure, the position of the IV bag and drip chamber may be reversed, i.e., the drip chamber is elevated above the IV bag to cause the air in the IV bag to move to the top to the location near the fluid port 174. With the tube set in that position, the valve may be opened allowing air to be expelled or purged from the IV bag and the tubing connecting the IV bag to the drip chamber. With essentially all of the air purged from the IV bag, the valve 300 is again closed to seal the fluid path and the tube set reoriented for normal use (e.g., with the IV bag over the drip chamber). Next, with the drip chamber in a normal operating orientation (e.g., with the neck portion oriented vertically or upward), the valve is again opened (e.g., by pressing the button) to fill or prime the drip chamber to the appropriate fill level and closed (e.g., by releasing the button) once the drip chamber has been primed. No pumping of the drip chamber is required and thus no additional air is forced into the IV bag, but instead the total amount of air remaining in the fluid path from the IV bag to the drip chamber volume is significantly reduced as compared to the starting point. The tube set may then be connected to the patient and the roller clamp may be used to adjust the drip rate to the desired rate. The tube set, and more specifically the drip chamber can be in any orientation during use without any added risk to the patient. Similarly, with pressurized infusion, the bag may be positioned anywhere, e.g., on the stretcher next to the patient's body, without increasing the risk to the patient. Beyond reducing air embolism in human patients, the tube sets described herein, particularly those used with a pressure delivery system (e.g., a pressure cuff with pressure regulator as described in the incorporated herein U.S. Ser. No. 16/093,552), may advantageously improve the delivery of fluids to non-human patients such as in the veterinary field, where confining the animal to a single location and maintaining the IV bag at an elevated position may be similarly problematic as in emergency response scenarios.

FIG. 18 shows another example of a drip chamber cap 820 that incorporates a priming apparatus according to the present disclosure. The drip chamber cap 820 may be provided as part of a modular system or kit 800 that includes a plurality of interchangeable components for assembling an IV infusion tube set. In some embodiments, the modular system 800 may include all of the components shown in FIG. 18, or a subset of the components shown therein. In some embodiments, the system 800 includes at least the component needed to assemble and provide a single functioning IV tube set.

In some embodiments, the modular system 800 may include at least one inflow member 810, at least one drip chamber cap 320, at least one filter supports for supporting and coupling a cap filter 840 to the drip chamber cap 820, at least one set of components for a fully assembled drip chamber 850, and at least one outflow member 860. In some embodiments, the modular system 800 includes multiple different inflow members, such as first inflow member 810-1 configured as an IV spike suitable for use with saline-based solutions, a second inflow member 810-2 configured as an IV spike suitable for use with colloid fluids, a third inflow member 810-3 configured as larger diameter extension tubing and a fourth inflow member 810-4 configured as smaller diameter extension tubing. The larger diameter extension tubing 810-3 may be suitable for use with larger subjects, such as in certain veterinary application (e.g., for equine or other larger animals), while the smaller extension tubing 810-4 may be suitable for use with smaller human or non-human subjects. Each of the inflow members 810 may be configured to interchangeably couple to the drip chamber cap 820. For example, each inflow members 810 may each have, at their proximal ends, a common coupling interface (e.g., a coupling fitting) for interchangeably coupling to the drip chamber cap 820. In some embodiments, the common interface is configured for insertion into the distal side of the drip chamber cap 820. The common interface for coupling to the drip chamber cap may be provided by external surface(s) of the proximal ends of the inflow member, while internal surfaces (i.e., inner diameter or other parameter) of the inflow members may be differently configured for different use cases. In some embodiments, the coupling interface may be configured for frictionally engaging (e.g., press fitting into) the distal side of the drip chamber cap 820. In other embodiments the coupling interface may include a threaded coupling. In some embodiments, the inflow member selected from the modular kit may additionally or alternatively be bonded to the drip chamber cap 820.

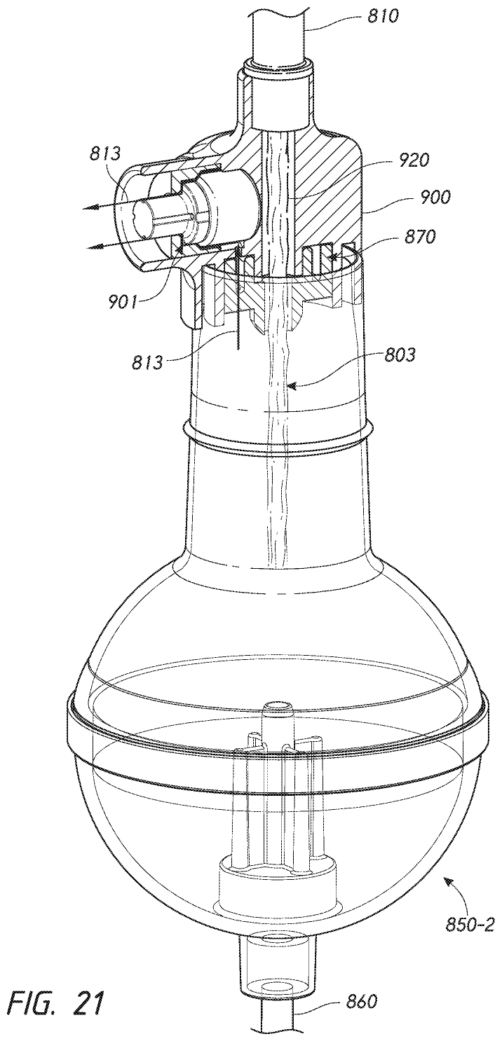

In some embodiments, the kit 800 includes a plurality of drip rate adjustors 830 each of which also functions as a filter support and may thus also be referred to as filter supports 830. Each of the filter supports (e.g., first filter support 830-1, second filter support 830-2, third filter support 830-3, and fourth filter support 830-4) includes a central through-passage that has a differently sized orifice and may thus provide a different drip rate (e.g., about 60 DPML, about 20 DPLM, about 15 DPLM and about 10 DPLM, respectively) into the drip chamber 850. The outlet orrifice a the filter support may be configured to have virtually any desired size and thus a drip chamber cap with virtually any desired drip rate may be provided with the interchangeably drip rate adjustor of the modular kit of the present disclosure. Each of the filter supports is configured to be assembles with a filter layer 840 into a filter assembly 870 (see FIG. 20), which is then coupled to the proximal side of the drip chamber cap 820 and is thus also referred to as cap filter assembly or simply cap filter.

The kit 800 may include at least one drip chamber 850, which may but need not be an all position drip chamber. The term all position drip chamber as used herein implies that the drip chamber can be properly used irrespective of the orientation of the drip chamber. That is, the drip chamber is specifically designed for intravenous fluid delivery irrespective of the orientation and/or position of the drip chamber relative to the patient. In some embodiments, the drip chamber 850 is assembled from multiple components (e.g., an upper portion and a lower portion), the components for at least one drip chamber may be included in a kit 800. The drip chamber 850 may be implemented according to any of the examples herein (e.g., drip chamber 100) or by any other suitable drip chamber currently existing or later developed. In some embodiments, the kit 800 includes multiple sets of upper and lower portions (e.g., two upper portions and two lower portions) that be interchanged to assemble drip chambers of multiple different configurations (e.g., four different configurations). For example, the kit 800 may include a first upper portion 852a (e.g., a rigid upper portion) and a second upper portion 852b, which may be formed, at least partially, of a flexible material and may thus be soft or squeezable. Each of the upper portions 852a and 852b are configured to interchangeably couple to a lower portion (e.g., to the first lower portion 854-1 and the second lower portion 854-2). In some embodiments, the kit includes a first lower portion 854-1 configured for use with blood-based products. The first lower portion 854-1 include a blood filter 858 provided, for example, across or over the outlet 859, as shown in the example in FIG. 18. In some embodiments, the kit 800 includes a second lower portion 854-2 be configured for use with non-blood-based fluids (e.g., a saline-based IV fluid) which need not include the blood filter. Each of the upper portions (e.g., first and second upper portions 852a and 852b) may couple to each of the lower portions (e.g., first and second lower portions 854-1 and 854-2) to provide a drip chamber 850 having one of four configurations. For example, either one of the first or second upper portions 852a and 852b may be coupled to the first lower portion 854-1 to provide either a soft top or a hard top drip chamber 850-1, depending on which of the upper portions was used, for infusion of blood-based products such as plasma. Similarly, either one of the first or second upper portions 852a and 852b may be coupled to the second lower portion 854-1 to provide either a soft top or a hard top drip chamber 850-2 suitable for infusion of non-blood-based products such as saline-based IV fluids. In some embodiments, the kit 800 includes only one upper portion (e.g., a rigid upper portion) and two lower portions. In yet other embodiments, the kit includes two upper portions but only a single lower portion (e.g., the second lower portion 854-2). In yet other embodiments, a kit includes only one upper portion (e.g., a rigid upper portion) and only one lower portion (e.g., a non-blood based lower portion).

The modular system 800 may further include a plurality of different outflow members 860. Similar to the extension tubing on the upstream side of the drip chamber, downstream tubing of different inner diameters may be provided, each of which may be suitable for a different application (e.g., medical or veterinary) and each of which is configured to interchangeably couple to the proximal side of the drip chamber for fluidly connecting the outlet 859 to the IV site in the subject. In FIG. 18 two different outflow members are shown, namely first larger diameter outflow member 860-1 and second smaller diameter outflow member 860-2 are shown. However, in other embodiments, fewer or more outflow members 860 may be included in a kit 800. The common coupling interface 862 of the outflow members 860 in this example are configured for insertion into and frictionally engaging the proximal side of the drip chamber 850. In other examples, a different interface for coupling (e.g., threadedly coupling and/or bonding) the outflow members to the drip chamber 850 may be used.

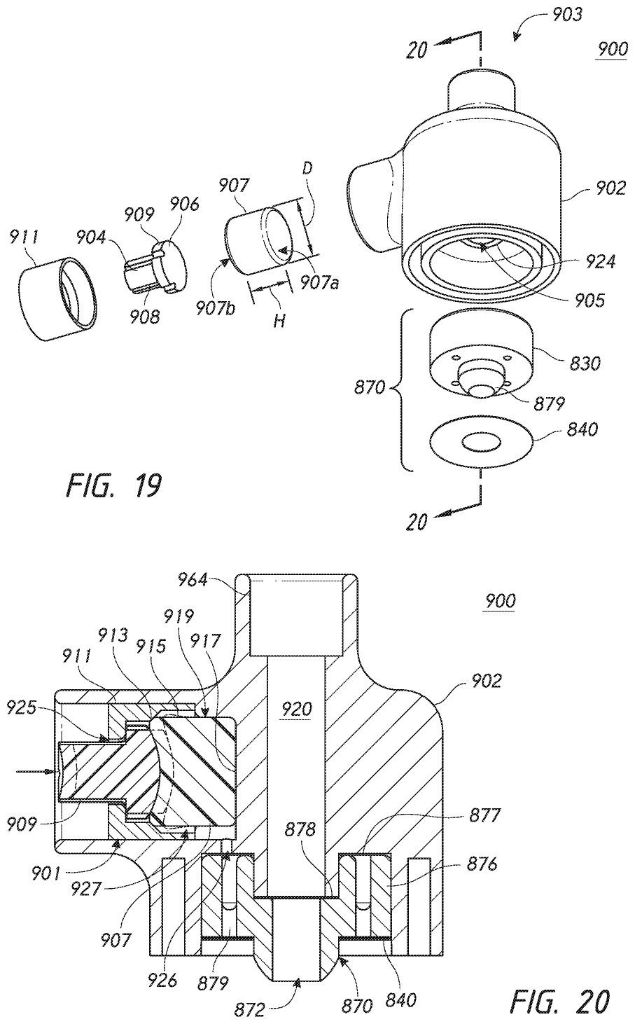

With further reference to FIGS. 19-21, a priming apparatus incorporated into a drip chamber cap 900 according to the present disclosure will be further described. The drip chamber cap 900 may be used to implement the drip chamber cap of an IV fluid infusion system according to any of the examples herein (e.g., drip chamber cap 820 of the modular system 800). Also, elements of the priming apparatus, such as the closure mechanism 901, may be used in place of the closure mechanism (e.g., valve actuator and seal of the valve 300) of any of the other examples of drip chamber caps (e.g., spike caps 201, 501, and 601 or cap 400) described herein.

In accordance with further examples of the present disclosure, an apparatus for priming a drip chamber, which in use is connected to an IV bag, includes a drip chamber cap 900, which may be implemented in part by a body 902 configured to mount to and cover the inlet of a drip chamber (e.g., drip chamber 850-2). In some embodiments, the body 902 may be integrally formed with drip chamber, which may obviate the need for a coupling interface between the body and the drip chamber. The drip chamber cap 900 (e.g., body 902) has a distal opening 903, a first proximal opening 905, and a fluid passage 920 connecting the distal opening 903 to the first proximal opening 905 to enable transmission of IV fluids from the IV bag into the drip chamber 850-2. The drip chamber cap 900 is coupled by its proximal side to the drip chamber 850-2 such that the first proximal opening 905 is in fluid communication with the drip chamber interior. The body 902 defines a vent cavity 927. The vent cavity 927 communicates with the interior of the drip chamber 950-2 via a second proximal opening 926 on the proximal side of the drip chamber cap 900 and also communicates with the ambient air via a vent outlet 925. As such, a secondary fluid passage, also referred to as vent passage is defined from the interior of the drip chamber 850-2 through the drip chamber cap 900 to the exterior (i.e., to ambient air) for drawing air out of the drip chamber interior during the priming process. A means (e.g., closure mechanism 901) is operatively associated with the vent cavity 927 and thus, the secondary fluid passage. The means (e.g., closure mechanism 901) is operated by user force to selectively open and close the vent outlet 925. When the means is in a closed position and the vent outlet 925 is, thus, closed or sealed, the vent cavity 927 is hermetically sealed from the ambient air, thus ambient air is prevented from entering the vent cavity 927, and consequently the fluid system (e.g., the drip chamber and/or the IV bag), through the cap 900 As such, the priming apparatus of the present disclosure can be used to purge substantially all of the air out of the IV bag, further reducing the risk of embolism.

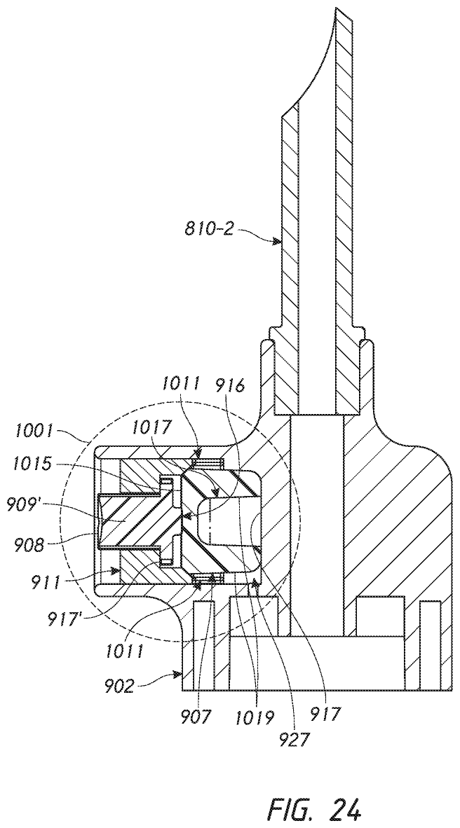

In some embodiments, the means (e.g., closure mechanism 901) for selectively opening and sealing the vent cavity 927 includes a seal (e.g., resilient member 907) positioned within the vent cavity 927. The closure mechanism 901 further includes an actuator (e.g., button 909) for temporarily move at least a portion of the seal away from the vent outlet 925 to open the vent outlet. The closure mechanism 901 (e.g., the seal and actuator) are biased toward the closed position. That is, the seal is biased toward the vent outlet sealing the vent outlet. In FIGS. 19-21, the seal is implemented by resilient member or body 907. The resilient member 907 may be implemented by a body having any suitable shape, such as by a substantially cylindrical body 915 as in the present example, a substantially spherical body, or a body having a different suitable shape (e.g., a cup-shaped body as in the example in FIG. 24). The resilient member 907 may be made of any suitable elastomeric material (e.g., silicone). The seal (e.g., resilient member 907) in this example extends from the base 917 of the cavity to the vent outlet. The actuator (e.g., button 909) may be arranged to engage (e.g., contact) the resilient member to compress the resilient member away from the vent outlet toward the base 917 of the vent cavity 927 (as shown in phantom line in FIG. 20). In this embodiment, the closure mechanism 901 is biased toward the closed position (e.g., the seal is biased toward the vent outlet 925) by the resilience (or spring force) of the resilient member 907. In other embodiments, the seal may be biased toward the vent outlet by a spring or other resiliently deformable member other than the seal itself, e.g., as shown in FIGS. 4-5. For example, in some such other embodiments, the seal may be implemented by an o-ring 319 and the actuator (e.g., button 310) may be configure the translate the o-ring toward and away from the vent outlet for opening and closing the vent outlet.

The seal and the actuator of the closure mechanism 901 may be retained within the cavity 927 by a retainer 911, shown here as a substantially annular structure. The annular retainer 911 may be configured to be press-fit and thus frictionally retained in the cavity 927. In other embodiments, the retainer 911 may be treaded and/or bonded to the body 902 so as to remain fixed relative to the cavity 927 during use. The vent cavity 927 may include a seat 919 into which the resilient member 907 is press-fit. The retainer 911, when press fit into the cavity 927 may abut against a shoulder below which the base 917 is recessed to define the seat 919. The resilient member 907 may be retained in the cavity 927 (e.g., in the seat 919) purely by frictional forces between resilient member 907 and the seat 919 or optionally additionally by being bonded to the cavity 927. In some embodiments, the cavity 927 may be wider from the shoulder toward the vent outlet 925 to accommodate the retainer 911 and/or any expansion of the resilient member 907 when under compression. The resilient member 907 is shown as a substantially cylindrical elastomeric body having a width, in this case a diameter D, and a height H defined between the first side 907a and the second side 907b of the resilient member 907. In other embodiments, the resilient member 907 may have a different shape (e.g., rectangular prism, frustoconical, or other suitable shape). When the actuator (e.g., button 909) is actuated to the open position, as shown in FIG. 20, such as by the button 909 pressing against the second side 907b of the resilient member 907, the resilient member 907 is compressed against the base 907 of the cavity 927, its height H slightly decreases to open a fluid path 813 out of the vent cavity 927. In some embodiments, the resilient member 907 may be at least partially compressed in its closed or nominal state to provide a desire amount of biasing force against the button in its resting (e.g., closed) state. In such examples, pressing of the button causes the resilient member 907 to compress or deform further from its partially compressed (or nominal) state.

As noted above, the retainer 911 may be implemented by an annular structure that substantially enclosed the vent cavity and that has central bore which defines the vent outlet. The actuator may be implemented by a button 909 having a base 906 and a post 908. The base 906 is wider than the post 908. In some embodiments, the base is wider than any other portion of the button 909. The base 906 is positioned within and retained inside the vent cavity 927 by the retainer 911 while the post 908 extends out of the vent cavity 927 through the central bore of the retainer 911 and vent outlet 925. The base 906 engages (e.g., by contacting) the seal (e.g., resilient member 907) for temporarily moving at least a portion of the seal away from the vent outlet 925. In use, the button 909 moves relative to the retainer 911. The button 909 is biased towards the exterior by the resilience of the seal member and moves, responsive to user force, in the opposite direction, against the biasing force of the seal to open the vent outlet. In some embodiments, the diameter of central bore of the retainer 911 may vary along the length of the bore. The central bore may be wider near the side of the retainer 911 facing the cavity 927 than near the side facing the exterior (i.e. ambient air). The central bore may have a first portion narrower than the base 906 but sufficiently wide to accommodate passage and the free movement of the post 908 therethrough. The bore may include a second portion adjacent to and wider than the first portion. The second portion may be sufficiently wide to accommodate the width of the base 906 such that the base can move freely into and out of the second portion. The bore may have a third portion adjacent to and wider than the second portion. A tapered surface may connect the second portion to the third portion of the central bore and provide the sealing surface 913 of the vent outlet 925. The seal may be sized to extend from the base 917 to the vent outlet and more specifically to the sealing surface 913 of the vent outlet 925. In this example, the cylindrical resilient member 907, in its nominal, substantially uncompressed state, has a diameter D corresponding to the width of the seat 917 and which is smaller than the diameter of the third portion of the central bore, and has a height H corresponding to the distance between the base 917 and the tapered sealing surface 913. The external portion of the button 909 that extends beyond the retainer 911 may be surrounded by a shroud configured to protect the button 909 from accidental actuation. In the example in FIGS. 19-21 is a substantially cylindrical enclosure surrounding all peripheral sides of the post 908. In some embodiments, one or more lengthwise grooves 904 may be formed along the exterior surface of the button 909 to facilitate the outflow of air from the cavity 927.

A filter assembly 870 may be coupled to the proximal side of the body 902. The filter assembly includes a filter layer 840 (e.g., a microbial filter). The filter layer 840 may have any suitable micron rating for filtering bacteria or other organisms. In some examples, the filter layer 840 may be a filter having 1 micron rating. In some embodiments, the filter layer 840 may be 1.2 micron filter. The filter layer 840 may be attached to a filter carrier or support 830. In the example in FIG. 20, the filter support 830 is received in an annular cavity 874 on the proximal side of the drip chamber cap 900. The annular cavity 874 surrounds an outlet tube 924 of the drip chamber cap 900 which defines the outlet 905 of the drip chamber cap 900. The filter support 830 includes a recess 878 on its distal side and a tubular extension 879 on its proximal side. A central through-passage 872 extends from the recess 878 on the distal to an opening at the proximal end of the tubular extension 879. The central through-passage 878 may be substantially aligned with the fluid passage 920 but may have a different diameter than the fluid passage 920 and may thus function as a drip rate adjustor, for example to decrease the drip rate into the drip chamber from the drip rate provided by the outlet tube 924. When the filter assembly 870 is coupled to the cap 900, the outlet tube 824 is received in the recess 878 of the filter assembly, while a substantially annular portion 876 of the filter support is received (e.g., frictionally engaging) the annular cavity 874. The filter support includes one or a plurality of openings 879 arranged radially around the central through-passage 872 on the proximal side of the filter support 830. The one or more openings 879 connect to an annular groove on the opposite distal side of the filter support 830. Air is communicated through the filter support 830 toward the second proximal opening 926 and into the vent cavity 927 through the one or more openings 879. In some embodiments, it may be desirable to reduce the rate of flow of air through and out of the vent cavity, and the size of the openings 879 and associated channels through the filter support 830 may be used to so limit the flow of air, which limits the drip chamber fill rate and may reduce the risk over filling the drip chamber. The filter layer 840 is arranged to cover each of the one or more openings 879 to maintain the interior of the fluid system sterile. On its proximal side, the body 902 may define a seat 964 for receiving the proximal end of an inflow member. The seat 964 may be aligned with the fluid passage 920 and is in fluid communication therewith.

FIG. 24 shows a priming apparatus 1001 according to further examples herein. Like other examples herein, the priming apparatus 1001 may be incorporated into a drip chamber assembly at a location proximate the drip chamber inlet. In FIG. 24, the body 902 is illustrated assembled with an inflow member, shown here as a colloid spike 810-2, operatively coupled to the coupling interface on (e.g., inserted into) the distal side of the body 902. In other embodiments, a different inflow member may be assembled to the body 902 or integrally formed therewith. In FIG. 25, the priming apparatus 1001 is shown accommodated in a cavity of the cap body 902. The priming apparatus 1001 may include similar components and function similarly to the priming apparatus described with reference to the example in FIGS. 19 and 20. For example, the priming apparatus 1001 in FIG. 24 includes a sealing member or simply seal, shown here as resilient body 907, which may be formed of any suitable elastomeric material (e.g., silicon, rubber or other elastomer). The priming apparatus also include an actuator (e.g., button 909′) that engages the seal (e.g., resilient body 907) to temporarily displaces at least a portion of the seal away from the vent inlet to provide the priming apparatus in an open position. The seal and actuator may be securely held in the cavity 927 by a retainer 911.

As described above, the vent outlet may be defined at least in part by the retainer 911, specifically by a central bore of the retainer 911. The central bore may have a cross-sectional geometry that cooperates with the cross-sectional geometry of the button 909′ passing through the retainer 911. Thus, while shown as having a substantially circular cross-section, the central bore of retainer 911 may have non-circular (e.g., a rectangular or triangular) cross section in other examples. The width (e.g., diameter) of the central bore of retainer 911 may vary along its length, as previously described. The retainer 911 defines a seal engagement surface against which the seal presses to seal the vent outlet. The seal engagement surface of the retainer 911 may be provided by a tapered surface (e.g., a substantially flat or curved surface) connecting the two adjacent portions of the central bore closest to the seal, as shown in FIG. 24. While a tapered seal engagement surface may provide for a more consistent and reliable sealing interface by providing a larger area of engagement, in some embodiments, the tapered surface may be omitted and the seal may instead press against the edge between the two portions of the central bore closest to the seal. In some embodiments, lengthwise grooves 1011 may be defined in the third portion of the central bore of the retainer to provide a path for the flow of air that remains substantially unobstructed, particularly when the seal is further compressed within the cavity 927 for opening the vent outlet.

The resilient body 907 is shown here as a substantially cup-shaped body having a substantially U-shaped cross-section defined by peripheral wall (or leg portions) 1019 and a central portion 1015. The resilient body 907 is received within a seat of the cavity 927 with the peripheral wall (or leg portions) 1019 provided against the base 917 of the cavity 927. The button 909′ engages the central portion 1015. Specifically, the button 909′ may include a central protrusion 916 extending from the side of the base 917′ facing the cavity. The base 917′ of the button 909′, and more specifically the central protrusion 916, presses against the central portion 1015 of the resilient body 907 to compress, responsive to user force, the resilient body 907 toward the base 917. When so compressed, the central portion 1015 may deform or displace towards the base 917, which causes the leg portions 1019 to deform or displace radially inward, breaking the seal between the resilient body 907 and the sealing engaging surface to open the vent outlet. The seal (e.g., resilient body 907) and/or actuator (e.g., button 909 or button 909′) may be configured differently in other embodiments. For example, the resilient body 907 may be substantially ball shaped (e.g., a substantially spherical resilient body), which may be seated in a seat or optionally retained within the cavity solely by the retainer 911. In yet other embodiments, the seal may be differently configured, for example having an accordion, baffle or other shape including one or more flexures along the height (H) of the resilient member to facilitate compression of the seal away from the vent outlet, which may in some such cases be formed of non-resilient material(s). The central bore of the retainer 911 may be suitably configured to accommodate, retain, and allow operative compression of seals having a variety of different shapes. In some embodiments, the seal (e.g., resilient member 907) may be retained within the cavity (e.g., by retainer 911) in a nominal state which may correspond to the resilient member's uncompressed state or to a state in which the resilient member is at least slightly compressed against the retainer to seal the vent outlet. Opening the vent outlet responsive to user force applied to the button compresses the seal, in some cases further from their nominal compressed state, and away from the vent outlet.

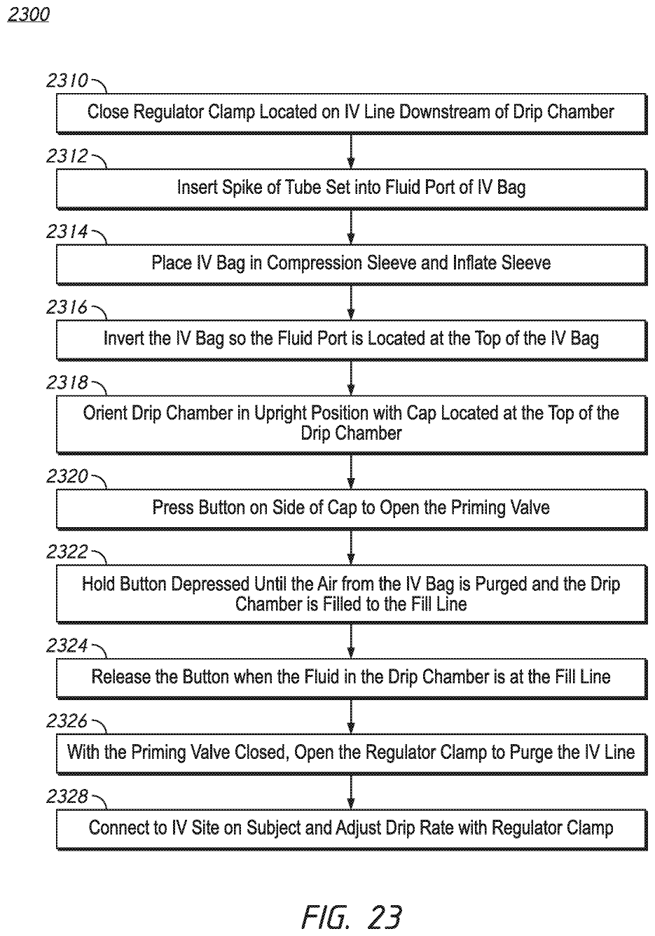

The assembly and operation of a fluid infusion system with a priming apparatus according to some embodiments herein will be described further with reference to the drip chamber assembly shown in the partial cutaway view in FIG. 21 and also with reference to the flow diagrams in FIGS. 22 and 23. FIG. 22 shows a flow diagram of a process 2200 for assembling an infusion tube set from a modular system or kit, according to some examples of the present disclosure. As described with reference to FIG. 18, the modular kit may include a plurality of different inflow members each of which may be interchangeably usable with a drip chamber cap that embodies a priming apparatus according to the present disclosure. The process 2200 may begin by selecting a filter assembly, as shown in block 2210. The filter support of each of the filter assemblies in a modular system may be configured to provide a different drip rate, thus the filter assembly may be selected based upon the desire drip rate. The selected filter assembly may be coupled to the bottom side of the drip chamber cap, as shown in block 2212, for example by press-fitting the filter support into an annular cavity on the bottom side of the cap. A drip chamber may be selected based upon the type of IV fluid to be administered with the tube set, as shown in block 2214. For example, for blood-based products, a drip chamber having a blood filter is selected and a drip chamber cap is installed over the inlet of the selected drip chamber, as shown in block 2216. The drip chamber cap may be a cap that incorporates a priming apparatus according to the any of the embodiments of the present disclosure. An inflow member is selected, as shown in block 2218, and operatively coupled to the inlet of the drip chamber cap, as shown in block 2220. In some embodiments, the selected inflow member is coupled to the drip chamber cap by inserting the proximal end of the inflow member into the distal side (e.g., into seat 964) of the drip chamber cap. If the selected inflow member is not integrated with a spike (see block 2221), e.g., if the selected inflow member is extension tubing, a spike is connected to the distal end of the inflow member, as shown in block 2222. An outflow member may be selected from a plurality of different outflow members (e.g., different diameter tubing), as shown in block 2224. One or more of the steps of process 2200 may be performed in different order, for example the step in block 2218 may be performed before the step in block 2216. Similarly the step in block 2224 may be performed before the step in block 2218. Steps may be reordered, combine, omitted or additional steps may be included to assemble an infusion tube set, e.g., as the one shown in FIG. 21.

FIG. 23 shows a flow diagram of a process for priming a drip chamber of an infusion tube set according to some examples of the present disclosure. The process 2300 may begin after an infusion tube set has assembled, at least partially, for example using the process 2200 described above. The process 2300 may begin by restricting the flow of the drip chamber, such as by closing the regulator clamp located on the proximal tubing downstream of the drip chamber, as shown in block 2310. Next, the spike cap is removed and the spike is inserted into a fluid port of the IV bag, as shown in block 2312. The spike bag may then be placed inside a compression sleeve, and the sleeve is inflated, as shown in block 2314, to apply compression on the bag to the appropriate (e.g., regulated) pressure. The external pressure applied to the bag via the compression sleeve may be regulated using a pressure regulator to an appropriate pressure (e.g., to about 100 mm Hg, 200 mm Hg or other pressure as may be appropriate depending on ambient pressure during IV therapy). As shown in block 2316, the IV bag is inverted so that the fluid port(s) is located at the top of the bag. The drip chamber is oriented in an upright position such that the cap with the priming apparatus is located at the top of the drip chamber, as shown in block 2318, and as illustrated in FIG. 21. While holding the drip chamber in the upright position, and as shown in block 2320, the priming apparatus is actuated to the open position, e.g., by pressing the button of the closure mechanism 901 integrated into the cap. In block 2322, the button is held in the depressed position until substantially all the air form the IV bag is purged out of the bag. The air from the bag passes into the drip chamber and then out of the drip chamber, through the vent path 813 in the cap, as fluid 803 (see FIG. 21) fills the drip chamber 850-2. The button is held depressed until the drip chamber is filled to the fill line, as shown in block 2322, which may be achieved in as short a time as a few seconds. As shown in block 2324, when the fluid in the drip chamber is at the fill line, the button is released, which hermetically seals the vent passage(s) in the drip chamber cap, hermetically sealing the drip chamber and the bag. With the priming valve closed, no air can enter the bag and the drip chamber. With the priming valve closed, the regulator clamp is opened to purge the IV line downstream of the drip chamber, as shown in block 2326, and the IV line can then be safely connected to the IV site on the subject and the roller clamp adjusted for the desired drip rate, as shown in block 2328.

This description of examples is provided to aid in understanding of the present disclosure. Each of the various aspects and features of the disclosure may advantageously be used separately in some instances, or in combination with other aspects and features of the disclosure in other instances. Accordingly, while the disclosure is presented in terms of examples, individual aspects of any example can be claimed separately or in combination with aspects and features of that example or any other example. This description of examples is neither intended nor should it be construed as being representative of the full extent and scope of the present disclosure. The present disclosure is set forth in various levels of detail in this application and no limitation as to the scope of the claimed subject matter is intended by either the inclusion or non-inclusion of elements, components, or the like in this description. The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. An apparatus for priming a drip chamber connected to an IV bag, the apparatus comprising:
a drip chamber cap configured to be coupled to the drip chamber to cover an inlet of the drip chamber, the drip chamber cap comprising a distal opening, a first proximal opening on a proximal side of the drip chamber cap, and a fluid passage connecting the distal opening to the first proximal opening for transmitting IV fluid into an interior of the drip chamber;
a vent cavity formed in the drip chamber cap and in fluid communication with a second proximal opening on the proximal side of the cap and a vent outlet open to ambient air, and
a means for selectively, by operation of user force, opening and sealing the vent outlet, wherein ambient air is prevented from entering the vent cavity when the means seals the vent outlet.

2. The apparatus of claim 1, wherein the means comprises a seal within the vent cavity and biased toward the vent outlet to seal the vent outlet, the means further comprising an actuator configured to temporarily move at least a portion of the seal away from the vent outlet to open the vent outlet.

3. The apparatus of claim 2, wherein the seal comprises an o-ring and wherein the actuator is configured to translate the o-ring away from the vent outlet to open the vent outlet.

4. The apparatus of claim 2, wherein the seal comprises a resilient member and wherein the seal is biased toward the vent outlet by a spring force of the resilient member.

5. (canceled)

6. The apparatus of claim 2, wherein the actuator is configured to temporarily move at least the portion of the seal away from the vent outlet by compressing the seal against a base of the vent cavity.

7. The apparatus of claim 2, wherein the vent outlet is defined by a central opening of an annular retainer enclosing the vent cavity, and wherein the actuator comprises a button that moves relative to the annular retainer, the button having an internal portion retained in the vent cavity by the retainer and an external portion located outside of the vent cavity.

8. The apparatus of claim 7, wherein the button includes a post that passes through the central opening, a base connected to a side of the post inside the cavity, the base being wider than the central opening, and wherein the base engages the seal for moving at least the portion of the seal away from the vent outlet to open the vent outlet.

9. The apparatus of claim 8, wherein the base is wider than any other portion of the button.

10. The apparatus of claim 8, wherein the central opening is provided by a bore having a variable diameter along a length of the bore.

11. The apparatus of claim 8, wherein the bore has a wider diameter portion that accommodates the base and a narrower diameter portion that accommodates the post.

12. The apparatus of claim 7, further comprising a shroud around the external portion of the button.

13. The apparatus of claim 7, wherein the button comprises at least one lengthwise vent groove along a surface of the button.

14. The apparatus of claim 7, wherein the seal comprises an elastomeric member extending from a base of the vent cavity to the annular retainer, the base of the button configured to press against the elastomeric member to compress the elastomeric member against the base of the vent cavity for opening the vent outlet.

15. An infusion tube set including the apparatus of claim 1, the drip chamber, and a cap filter assembly coupled to the proximal side of the drip chamber cap.

16. The infusion tube set of claim 15, wherein the cap filter assembly is removably coupled to the drip chamber cap in interchangeable with another cap filter assembly of a plurality of cap filter assemblies, each having a different configuration.

17. The infusion tube set of claim 15, wherein the cap filter assembly is received within an annular cavity on the proximal side of the drip chamber cap.

18. The infusion tube set of claim 15, wherein the drip chamber is configured to be used for intravenous fluid delivery in any orientation of the drip chamber.

19. A modular kit comprising the apparatus of claim 1, the drip chamber, and a plurality of cap filter assemblies, wherein each of the plurality of cap filter assemblies has differently sized outlet orifice, and each of the plurality of cap filter assemblies are configured to removably interchangeably couple to the proximal side of the drip chamber cap, the modular kit further comprising a plurality of inflow members, each of the plurality of inflow members having a different configuration, and each of the plurality of inflow members being configured to removably interchangeably couple to the distal side of the drip chamber cap.

20. The modular kit of claim 19, wherein a proximal end of each of the plurality of inflow members is configured to be inserted into and frictionally engage a seat at the distal opening of the drip chamber cap.

21. The modular kit of claim 20, wherein a first one of the plurality of inflow members comprises a blunt distal end and a second one of the plurality of inflow members comprises a spike at its distal end.

22. An apparatus for priming a drip chamber of an infusion tube set, the apparatus comprising:
a body having an inlet, an outlet, and a fluid passage connecting the inlet to the outlet, wherein the body is configured to be coupled to a drip chamber to position the outlet in fluid communication with an interior of the drip chamber;
a valve received in a cavity of the body, wherein the valve cavity is in fluid communication with the interior of the drip chamber via a valve inlet and with ambient air via a valve outlet, and wherein the valve is configured to be actuated by a user to selectively open and seal the valve outlet, wherein the valve cavity is hermetically sealed from the ambient air when the valve outlet is sealed.

23. The apparatus of claim 22, wherein the valve comprises a sealing member configured to press against the valve outlet to seal the valve outlet, and an actuator configured to temporarily displace the sealing member away from the valve outlet to open the valve.

24. The apparatus of claim 23, wherein the actuator comprises a button having a base movably received within the cavity and a post narrower than and extending from the base and through the valve outlet.

25. (canceled)

26. The apparatus of claim 24, wherein the seal engages a side of the base facing away from the valve outlet.

27. (canceled)

28. The apparatus of claim 26, wherein the button is biased toward the closed position by the seal.

29-31. (canceled)

32. A fluid infusion set comprising:
a drip chamber having a drip chamber inlet for providing a fluid into an interior of the drip chamber;
a cap covering the drip chamber inlet, wherein the cap comprises a cap body defining a cap inlet on a distal side of the cap body, a cap outlet on a proximal side of the cap body and a fluid passage extending through the cap body and connecting the cap inlet to the cap outlet;
a valve received within a cavity in the cap body, wherein the cavity communicates with the interior of the drip chamber via a valve inlet opening on the proximal side of the cap body and wherein the cavity communicates with ambient air via a valve outlet, and wherein the valve includes a closure mechanism actuatable by a user between an open position in which air is permitted to pass through the valve outlet and a closed position in which the cavity is hermetically sealed from the ambient air.

33. The fluid infusion set of claim 32, wherein the drip chamber is configured to be used for intravenous fluid delivery in any orientation of the drip chamber.

34-36. (canceled)

37. The fluid infusion set of claim 32, wherein the closure mechanism comprises a button and a seal configured to seal the valve outlet when the closure mechanism is in the closed position, and wherein the seal is positioned between a base of the button and the valve outlet.

38. The fluid infusion set of claim 32, wherein the closure mechanism comprises a button and a seal configured to seal the valve outlet when the closure mechanism is in the closed position, where the button compresses the seal in a direction away from the valve outlet when the valve is in the open position.

39-43. (canceled)

44. The fluid infusion set of claim 32, further comprising an antimicrobial filter at the valve inlet opening, wherein the cap body defines an annular bore on the proximal side of the cap body, and wherein the filter is provided in the annular bore.

45. The fluid infusion set of claim 32, further comprising an antimicrobial filter at the valve inlet opening, wherein the filter is attached to a filter support received at least partially within the annular bore.

46. (canceled)
